(19) United States
(12) Patent Application Publication
(10) Pub. No.: US 2005/0169940 A1
(43) Pub. Date: Aug. 4, 2005

(54) Recombinant poxvirus not having a functional 3beta-hsd gene

(76) Inventors: Geoffrey Lilley Smith, London (GB); Jeffrey Bernard Moore, Brookline, MA (US); Patrick Clarke Reading, Melbourne (AU)

Correspondence Address: Leon R Yankwich;Yankwich & Associates, 201 Broadway, Cambridge, MA, 02139, US

(21) Appl. No.: 10/520,371

(22) PCT Filed: Jul. 4, 2003
(86) PCT No.: PCT/GB03/02909

(30) Foreign Application Priority Data
Jul. 5, 2002 (GB) 0215621.4

(51) Int. Cl. A61K 39/275; A61K 39/285; C12N 7/00; C07K 14/07

(52) U.S. Cl. 424/232.1; 435/235.1; 530/350

(57) ABSTRACT

A recombinant poxvirus, wherein the poxvirus genome does not comprise a functional gene encoding a 3β-hydroxysteroid dehydrogenase/Δ⁵-Δ⁴isomerase, may be used for vaccination against infection by a poxvirus. The recombinant poxvirus in which, additionally, the poxvirus genome comprises a non-poxvirus gene or a fragment of a non-poxvirus gene which gene or fragment encodes an antigen, for example, of a pathogenic agent, may be used to induce an immune e response against the antigen, for example, for vaccination against infection by the pathogenic agent.

The present invention relates to poxviruses with improved properties for use as a vaccine and to methods associated therewith.

### BACKGROUND

In the mid to late 20^{th} century, vaccinia virus (VV) was used to vaccinate large numbers of humans against variola virus, the causative agent of smallpox. The vaccination campaign was highly successful and smallpox was declared as eradicated by WHO in 1980. The success of VV as a live vaccine was due, in part, to its low production cost and the ability to administer the vaccine by simple dermal abrasion. Those advantages make VV and other poxviruses attractive for use as the basis for recombinant viral vaccines and, consequently, VV is currently the most explored recombinant viral vaccine (see, for example, Smith et al., 1983, Panicali et al., 1983, Moss et al., 1996, and Dorrell et al., 2001).

The genome of VV strain Copenhagen has been sequenced (Goebel et al., 1990) and it was found to comprise in the region of 200 genes. Of those genes, at least one third are dispensible for virus replication in vitro (Perkus et al., 1991). VV possesses several genes which have been proposed to aid evasion or suppression of the host immune system.

A VV open reading frame (ORF) denoted as SalF7L in Western Reserve (WR) strain and as A44L in Copenhagen strain encodes a protein that has 31% sequence identity to human 3β-hydroxy steroid dehydrogenase/Δ⁵-Δ⁴isomerase (hereinafter 3β-HSD) (Goebel et al. 1990, Blasco et al., 1991, and Smith et al., 1991). The SalF7L/A44L (hereinafter referred to as A44L) gene product was shown to have activity as a 3β-HSD by the conversion of pregnenolone to the steroid hormone progesterone (Moore and Smith, 1992). Deletion of the gene was shown to abrogate virus 3β-HSD activity and reduce virus virulence. For example, mice infected with VV WR lacking A44L showed reduced mortality and a milder weight loss compared to those infected with wild-type VV WR.

Various roles for the A44L 3β-HSD in VV virulence have been proposed. Proposals include that the steroid hormone product increases the metabolic rate, increases cell proliferation or causes immunosuppression. None of those effects has yet been proven.

The role of A44L in VV virulence and immunogenicity was investigated by Sroller (Sroller et al., 1998). It was observed that only moderate attenuation of virulence was achieved by deletion of A44L and that immunogenicity of the VV was not affected. It was concluded that a role for A44L in immunosuppression was unlikely as far as the humoral immune response is concerned.

3β-HSD Enzymes

The 3β-hydroxysteroid dehydrogenase/Δ⁵-Δ⁴isomerase (3β-HSD) isoenzymes play a key role in cellular steroid hormone synthesis. The 3β-HSD enzyme catalyses the conversion of Δ⁵-3β-hydroxysteroids to Δ⁴-3-ketosteroids, a reaction that is required for the biosynthesis of all classes of steroid hormones: progesterone, mineralocorticoids, glucocorticoids (GCs), androgens and estrogens. The enzyme is expressed in classical steroidogenic tissues (adrenal cortex, ovaries and testis) but also in peripheral tissues including the skin, liver, kidney and lung (Labrie et al., 1992; Martel et al., 1994; Pelletier et al., 1992). Multiple isoforms of 3β-HSD have been identified in human, rat and mouse tissues (Morel et al., 1997).

Steroids play an important role in the differentiation, growth and physiology of many mammalian tissues. In addition, steroids have multiple effects within the immune system. GCs are potent immunosuppressive and anti-inflammatory agents and are used therapeutically in the treatment of organ transplantation, autoimmune disease and a broad spectrum of inflammatory diseases (Boumpas et al., 1993). Although the precise mechanisms underlying their immunosuppressive effects have not been fully elucidated, GCs affect the immune system by modulating cytokine production, antigen presentation, and the migration and cytotoxicity of immune cells (Ashwell et al., 2000; Boumpas et al., 1991; Schwiebert et al., 1996). In addition, sex hormones are reported to modulate immune responses and have been implicated in sex-associated susceptibilities to infectious agents, for example coxsackievirus (Huber et al., 1999; Huber and Pfaeffle, 1994). GCs appear to have the potential to affect multiple aspects of the antiviral immune response.

GC release through the hypothalamic-pituitary-adrenal (HPA) axis occurs as part of the circadian rhythm (Dhabhar et al., 1994) and is induced by additional stimuli including physical or cognitive stress (Khansari et al., 1990) and cytokine responses to bacterial LPS (Fong et al., 1989; Imura and Fukata, 1994) or viruses such as murine cytomegalovirus (MCMV) (Ruzek et al., 1997).

Steroid hormones such as GCs have long been known to affect the pathogenesis of bacterial, protozoan and viral infections (Kass and Finland, 1953). A natural example of this is the severity of variola virus infections observed in pregnant women, who were more likely to exhibit the fatal haemorrhagic form of smallpox than were men or non-pregnant women (Rao et al., 1963), presumably due to the effect of pregnancy hormones. This could be partially mimicked by the administration of steroids such as cortisone to variola virus-infected macaque monkeys (Rao et al., 1968). Cortisone administration has also been shown to increase the severity of the primary inoculation lesion and to delay healing in guinea pigs (Kligman, 1951) and rabbits (Bugbee et al., 1960) infected with VV. Early leukocyte infiltration was also reduced in rabbits inoculated with VV (Bugbee et al., 1960).

Biochemically, mammalian 3β-HSD functions at multiple steps in steroid biosynthesis. While GCs are generally considered to be immunosuppressive, other steroids such as androstenediol (a metabolite of dehydroepiandrosterone (DHEA)) have been shown to augment immune responses to infections with coxsackievirus (Loria and Padgett, 1992), influenza virus (Padgett et al., 1997) or herpes simplex type 1 virus (Daigle and Carr, 1998).

A number of proteins are secreted from poxvirus-infected cells that can bind and inhibit specific components of the host immune system including IFNs, complement, cytokines and chemokines (Alcamí and Koszinowski, 2000). A44L is an intracellular v3β-HSD encoded by VV strain WR that increases virulence in vivo following intranasal (Moore and Smith, 1992; Sroller et al., 1998) infection of mice.

### SUMMARY OF THE INVENTION

The invention provides, inter alia, a recombinant poxvirus, wherein the poxvirus genome does not comprise a functional gene encoding a 3β-hydroxysteroid dehydrogenase/Δ⁵-Δ⁴isomerase, for use as a medicament. The invention also provides a recombinant poxvirus having a genome comprising a non-poxvirus gene or a fragment of a non-poxvirus gene which gene or fragment encodes an antigen, wherein the poxvirus genome does not comprise a functional gene encoding a 3β-hydroxysteroid dehydrogenase/Δ⁵-Δ⁴isomerase, for use as a medicament.

Preferably, the recombinant poxvirus of the invention is for use as a vaccine.

### BRIEF DESCRIPTION OF THE FIGURES

FIG. 1 shows the results of an assay of 3β-HSD activity in cells infected with various VV strains;

FIG. 2 shows the attenuation of VV infection by the deletion of A44L;

FIG. 3 shows the titres of vA44L (a wild-type VV expressing gene A44L), vΔA44L (a VV mutant lacking 86% of the A44L gene) and A44L-rev (a revertant virus in which the A44L gene has been re-inserted at its natural site into the vΔA44L deletion mutant) VV in the lungs (A), brains (B), spleens (C) and livers (D) of mice infected with those viruses;

FIG. 4 shows a characterization of bronchoalveolar lavage (BAL) cell suspensions from mice infected with vA44L, vΔA44L or vA44L-rev VV;

FIG. 5 shows an analysis of lymphocytes from BAL of VV-infected mice;

FIG. 6 shows the production of interferon (IFN)-γ in the lungs of VV-infected mice;

FIG. 7 shows the activity of virus-specific cytotoxic T lymphocytes (CTLs) in the lung of VV-infected mice.

FIG. 8 shows corticosterone levels in plasma and lungs after intranasal infection with vv.

### DETAILED DESCRIPTION OF THE INVENTION

The invention is based on the observation that the A44L gene of VV interferes with events in the host cellular response to VV infection. The applicants have now found that the absence of A44L in VV is associated with relatively mild weight loss and signs of illness, as well as reduced virus titres in lungs and secondary sites of virus replication (brain, spleen and liver). The absence of A44L is also associated with a more vigorous inflammatory response characterised by more rapid recruitment of CD4⁺ and CD8⁺ lymphocytes, enhanced IFN-γ production and augmented CTL activity in the lungs of infected mice. Furthermore, levels of corticosterone, the natural murine glucocorticoid (GC), were lower in the plasma and lungs of mice infected with VV lacking A44L in comparison with VV with A44L suggesting that enhanced steroid production by the A44L protein contributes to immunosuppression during VV infection.

The interference by A44L reduces the host's cellular immune response to VV, thus allowing protracted virus replication and improved virus dissemination in the host. Conversely, removal of the A44L gene leads to an increase in host cellular immune response to VV infection. In a vaccine, a strong host cellular immune response is desirable if effective immunity is to be developed.

The invention provides a recombinant poxvirus, wherein the poxvirus genome does not comprise a functional gene encoding a 3β-hydroxysteroid dehydrogenase/Δ⁵-Δ⁴isomerase, for use as a medicament. Preferably, the recombinant poxvirus of the invention is for use as a vaccine against a disease caused by a poxvirus.

Preferably the recombinant poxvirus is an orthopoxvirus or a derivative thereof. More preferably, the recombinant poxvirus is a VV, a cowpox virus, a camelpox virus or an ectromelia virus or a derivative of any of those viruses. Most preferably, the recombinant poxvirus is a VV. A VV may be a VV strain selected from the group consisting of Lister, Copenhagen, Wyeth, New York City Board of Health, NYVAC, Praha virus, DRYVAX Wyeth-derived virus, LIVP, IHD-J, IHD-W, Tian Tan, Tashkent, King Institute, Patwadanger, EM-63, Evans, Bern, LC16m0 or MVA. Preferably, the recombinant poxvirus is a VV strain selected from the group consisting of MVA, Lister, Copenhagen or Wyeth.

The medicament may be for use as a vaccine against a disease caused by an orthopoxvirus infection in man, i.e., in a human. The disease caused by an ortho-poxvirus is especially one selected from the group consisting of smallpox, monkeypox and cowpox. The medicament is especially for use as a vaccine against smallpox.

The recombinant poxvirus according to the invention also finds application in the veterinary field. The medicament may be for use as a vaccine against a disease caused by an orthopoxvirus infection in an animal, in particular in a mammal, for example, a non-rodent mammal. The animal may be, for example, a companion animal, an animal used in animal husbandry, or an animal used in sport or for transport, for example, a cat or dog, a member of the cattle family, a sheep or goat, a pig, a horse, or a member of the camel family. The disease caused by an orthopoxvirus in an animal is especially one selected from the group consisting of monkeypox, cowpox, and camelpox. For example, the medicament may be for use in the immunisation of cats against cat pox, mice against ectromelia, members of the camel family against camelpox, a wide range of mammals, for example rodents, cats, cows and other cattle, large felines or elephants against cowpox, or rodents or monkeys against monkeypox.

Alternatively, the recombinant poxvirus of the invention may be selected from the group consisting of parapoxviruses, avipoxviruses, suipoxviruses, molluscipoxviruses and yatapoxviruses. Sequences of capripoxviruses and leporipoxviruses that have been determined to date do not indicate the presence of a gene encoding a 3β-HSD. The amino acid similarity of the 3β-HSD enzymes between the genera is not particularly high, typically 40-45% identity. For the definition of the term “gene encoding a 3β-HSD” used herein, see below.

Preferably, such a recombinant poxvirus of the invention may be for use as a vaccine against a disease caused by a poxvirus, for example a molluscum contagiosum virus, infection in man, that is to say, in a human.

Alternatively, such a recombinant poxvirus of the invention may be for use as a vaccine against a disease caused by a poxvirus infection in an animal, especially a mammal, wherein the poxvirus is selected from the group consisting of parapoxviruses, avipoxviruses, suipoxviruses, molluscipoxviruses and yatapoxviruses. The animal may be, for example, as described above, for example, a non-rodent mammal. The animal may be, for example, a companion animal, an animal used in animal husbandry, or an animal used in sport or for transport, for example, a cat or dog, a member of the cattle family, a sheep or goat, a pig, a horse, or a member of the camel family.

Optionally, the recombinant poxvirus of the invention has a genome comprising a non-poxvirus gene or a fragment of a non-poxvirus gene which gene or fragment encodes an antigen.

The invention further provides a vaccine composition comprising a poxvirus according to the invention and a pharmaceutically suitable carrier.

The vaccine composition may comprise one or more additives selected from the group comprising an antibiotic, a preservative, a stabiliser and an adjuvant. Preferably, the vaccine composition according to the invention comprises one or more additives, for example a preservative and/or a stabiliser. The immunising effect of an immunogen in a vaccine may be enhanced by the addition of an adjuvant. An adjuvant co-stimulates the immune system in an unspecific manner causing a stronger specific immune reaction against the immunogenic determinant in the vaccine. The invention also provides a vaccine kit comprising a vaccine composition according to the invention.

The invention further provides a method of vaccinating a subject comprising administering to the subject an effective amount of an immunogenic agent, wherein the immunogenic agent is a poxvirus according to the invention or a vaccine composition according to the invention. The vaccination generally induces an immune response to the poxvirus used as immunogenic agent and hence provides protection against infection by the poxvirus or an immunogenically cross-reacting poxvirus.

The term “subject” is used herein to denote a human or a non-human animal. A non-human animal is, in particular, a mammal and may be a non-rodent mammal. The animal may be, for example, a companion animal, an animal used in animal husbandry or an animal used in sport or for transport, for example, a cat or dog, a member of the cattle family, a sheep or goat, a pig, a horse, or a member of the camel family.

The term “effective amount” denotes an amount effective to achieve the desired result.

The invention also provides the use of a recombinant poxvirus having a genome which does not comprise a functional gene encoding a 3β-hydroxysteroid dehydrogenase/Δ⁵-Δ⁴isomerase for the manufacture of a vaccine for the immunoprophylaxis of an infection caused by a poxvirus.

The terms “functional gene encoding a 3β-hydroxysteroid dehydrogenase/Δ⁵-Δ⁴isomerase” and “functional gene encoding a 3β-HSD” are used herein to mean a gene that gives rise to a gene product that has effective 3β-HSD activity.

A 3β-HSD, or 3β-hydroxysteroid dehydrogenase/Δ⁵-Δ⁴isomerase is any enzyme that catalyses the conversion of a Δ⁵-3β-hydroxysteroid to a Δ⁴-3-ketosteroid, irrespective of the amino acid sequence of the enzyme. The 3β-HSD activity in infected cells may be assessed, for example, using the tritiated pregnenolone method described by Moore and Smith (Moore and Smith, 1992). A level of activity in the assay statistically significantly higher than that in an uninfected cell is considered to be “effective” and hence is the activity level above which a gene encoding a 3β-HSD is considered to be “functional” for present purposes. Preferably, an effective level of 3β-HSD activity is more than twice the level in an uninfected cell. More preferably, an effective level of 3β-HSD activity is more than four times the level in an uninfected cell.

The absence of a “functional gene encoding a 3β-HSD” may be brought about by the poxvirus having no coding sequence for a 3β-HSD. Alternatively, it may be brought about by the poxvirus 3β-HSD gene coding sequence being disrupted, mutated or truncated such that its gene product has reduced activity or no activity. For example, 86% of the ORF may be lacking. As a further alternative, one or more mutations or deletions in the promoter or other upstream sequences may cause expression of the gene to be compromised, leading to reduced levels of gene expression or no gene expression.

Preferably, the absence of a functional gene encoding a 3β-HSD is brought about by removal or disruption of a gene encoding a 3β-HSD in a virus, the parental strain of which comprises a functional gene encoding a 3β-HSD.

An example of a gene encoding a 3β-HSD is the A44L gene of VV strain Copenhagen. The sequence of the A44L gene is given in the complete sequence of VV strain Copenhagen (Genbank Ref NC_{—}001559 and Goebel et al., 1990). The A44L gene of VV strain Copenhagen is in the Genbank sequence under accession number gi:9790357. The A44L ORF in VV strain WR is located in the F fragment of the SalI-restriction map of the virus genome and hence was originally designated as SalF7L. Hereinafter it is referred to as A44L (according to the HindIII-restriction map) in line with VV strain Copenhagen (Goebel et al., 1990).

Genes that encode, or that have putatively been assigned as encoding, a 3β-HSD have been identified in the genomes of other orthopoxviruses and in chordopoxviruses of genera other than orthopoxvirus. Only in the cases of the capripoxviruses and the leporipoxviruses sequenced to date has a gene encoding a 3β-HSD not been found in a parental poxvirus genome. The degree of amino acid identity between the proteins encoded by genes considered or shown to encode a 3β-HSD in the various genera and the A44L gene of VV strain WR is somewhat variable. Typically, the sequence of a 3β-HSD has amino acid identity of 25% or greater with the coding sequence of the protein encoded by the A44L gene. For example, the sequence of a 3β-HSD has amino acid identity of 35% or greater with the amino acid sequence of the protein encoded by the A44L gene. Most preferably, the sequence of a 3β-HSD has amino acid sequence identity of 40% or greater with the sequence of the protein encoded by the A44L gene.

By “derivative” of a particular virus is meant any virus that is derived from the particular virus. A derivative may be obtained by repeated passaging of the particular virus. Alternatively, a derivative may be obtained by site directed or random mutagenesis of the particular virus. A derivative generally has most of the characteristics and most of the genes of the particular virus from which it is derived. Typically, its genome has 90% sequence identity with the genome of the particular virus. For example its genome has 95%, optionally 98% sequence identity with the genome of the particular virus.

The invention also provides a recombinant poxvirus having a genome comprising a non-poxvirus gene or a fragment of a non-poxvirus gene which gene or fragment encodes an antigen, wherein the poxvirus genome does not comprise a functional gene encoding a 3β-hydroxysteroil dehydrogenase/Δ⁵-Δ⁴isomerase for use as a medicament.

Preferably, the recombinant poxvirus of the invention is one wherein the poxvirus is an orthopoxvirus or a derivative thereof. Preferably the recombinant poxvirus is one wherein the poxvirus is a VV, a cowpox virus, a camelpox virus or an ectromelia virus, or a derivative of any of those viruses. Most preferably, the recombinant poxvirus is a VV. A VV strain may be selected from the group consisting of Lister, Copenhagen, Wyeth, New York City Board of Health, NYVAC, Praha virus, DRYVAX Wyeth-derived virus, LIVP, IHD-J, IHD-W, Tian Tan, Tashkent, King Institute, Patwadanger, EM-63, Evans, Bern, LC16m0 and MVA. For example, the VV strain is selected from the group consisting of MVA, Lister, Copenhagen or Wyeth.

Alternatively, the recombinant poxvirus of the invention may be selected from the group consisting of parapoxviruses, avipoxviruses, suipoxviruses, molluscipoxviruses and yatapoxviruses.

The non-poxvirus gene or gene fragment that encodes an antigen may be any non-poxvirus gene or gene fragment against the gene product of which a protective immune response in a subject is desirable.

By “non-poxvirus gene” is meant herein a gene not belonging to a poxvirus of the genus of the poxvirus in question. For a given recombinant poxvirus, the non-poxvirus gene may be a gene belonging to a poxvirus of a different genus. Preferably, the non-poxvirus gene is a gene not belonging to any poxvirus.

The recombinant poxvirus of the invention is preferably for use as a vaccine for the prophylaxis of an infection caused in a subject by a pathogenic agent.

Preferably, the gene or gene fragment that encodes an antigen may be any non-poxvirus gene or gene fragment against the gene product of which a cellular immune response in a subject is desirable. Suitable genes include those encoding immunogenic peptides or polypeptides of an infectious pathogen, for example, for use in humans, an influenza virus, malaria, HIV, heptitis C virus, hepatitis B virus, herpes virus, a parasitic pathogen, for example tuberculosis or Leishmaniasis, a protozoan, for example a protozoan that causes ameobic dysentery. For use in animals, appropriate pathogen immunogenic peptides are known to those skilled in the art.

The recombinant poxvirus of the invention may be for use as a vaccine for the prophylaxis or treatment of a disease associated with aberrant cells. In such a recombinant poxvirus, the gene or gene fragment that encodes an antigen may be any non-poxvirus gene or gene fragment encoding an antigenic peptide or an antigenic polypeptide of aberrant cells, for example cancer cells, the elimination or induced quiescence of which is beneficial.

In some instances, it is beneficial for the whole gene that encodes an antigen to be present in the virus. In some cases, however, a fragment of the gene will suffice. In the case of a gene fragment, a gene product smaller than that of the whole gene is produced, which smaller gene product is or comprises an epitope or epitopes of the antigen in question.

The invention also provides a vaccine composition comprising a poxvirus of the invention and a pharmaceutically suitable carrier. The vaccine composition may optionally comprise an adjuvant.

The invention further provides a method of vaccinating a subject comprising administering to the subject an effective amount of an immunogenic agent, wherein the immunogenic agent is a poxvirus according to the invention or a vaccine composition according to the invention. The vaccination generally induces an immune response to the poxvirus used as immunogenic agent and hence provides protection against infection by the poxvirus or an immunogenically cross-reacting poxvirus.

The invention further provides a method of inducing a protective immune response in a subject comprising administering to the subject an immunogenic agent, wherein the immunogenic agent is a poxvirus or a vaccine composition according to the invention. Preferably, the immune response includes a cellular immune response. A protective immune response is induced to the poxvirus used as immunogenic agent and hence provides protection against infection by the poxvirus or an immunogenically cross-reacting poxvirus.

The invention also provides the use of a recombinant poxvirus according to the invention for the manufacture of a vaccine for the prophylaxis of an infection caused by a pathogenic agent wherein said poxvirus has a genome comprising a non-poxvirus gene or a fragment of a non-poxvirus gene which gene or fragment encodes an antigen of the pathogenic agent.

The invention also provides the use of a recombinant poxvirus according to the invention for the manufacture of a vaccine for the prophylaxis or treatment of a disease associated with aberrant cells, wherein said poxvirus has a genome comprising a non-poxvirus gene or a fragment of a non-poxvirus gene which gene or fragment encodes an antigen of the aberrant cells comprising the gene product of the said non-poxyiral gene.

A recombinant poxvirus of the invention may be prepared by methods known in the art (see for example Boyle, D. B. and Coupar, B. E. H., Gene, 1988, 65, 123-128). For example, a poxvirus lacking a functional gene that encodes a 3β-HSD may be produced by transfection of cells that had previously been infected with a poxvirus with a plasmid, the plasmid comprising DNA sequences homologous to sequences of the poxvirus flanking the gene that encodes a 3β-HSD or in the gene that encodes a 3β-HSD together with a selectable marker. After transfection and virus multiplication, recombinant viruses are selected using the selectable marker.

As mentioned above, the poxvirus of the invention may comprise a gene that encodes an immunogen. The gene encoding the antigen is introduced into the poxvirus in a manner known in the art. For example, a plasmid may be used that comprises the gene together with DNA sequences homologous to sequences of the poxvirus genome such that homologous recombination can take place between the plasmid and the genomic DNA. Preferably, the sequence of the poxvirus genome is one that may be disrupted without compromising the viability of the poxvirus. The homologous DNA sequences are preferably of sufficient length to enable homologous recombination between the plasmid DNA and the virus genomic DNA to take place. Accordingly, the DNA sequences preferably have a length of from 20 to 1000 bp. More preferably, the sequences have a length of from 100 to 800 bp. Still more preferably, the sequences have a length of from 300 to 500 bp. Sequences are considered homologous if they have 85% or more sequence identity. Preferably, homologous sequences have 90% or more sequence identity, more preferably, homologous sequences have 95% or more sequence identity, for example, 98% or more sequence identity. Most preferably, the homologous sequences used are identical to the genomic sequences in the virus.

By use of a plasmid comprising the gene encoding the antigen flanked by DNA sequences with homology to sequences of the poxvirus flanking the gene that encodes a 3β-HSD or in the gene that encodes a 3β-HSD A44L gene, it is possible to carry out the disruption of the gene that encodes a 30-HSD and the introduction of the antigen gene in a single step.

The gene encoding the antigen of interest is preferably engineered to be associated with transcriptional regulatory sequences for expression of the gene by poxvirus in an infected host cell. Such regulatory sequences preferably include a promoter from a poxvirus and a poxvirus termination sequence. Most preferably, a promoter from VV or a related poxvirus and a VV termination sequence are included. Most preferably, the VV termination sequence is a VV early transcriptional termination sequence (TTTTTNT).

The invention further provides a recombinant poxvirus having a genome comprising a non-poxvirus gene or a fragment of a non-poxvirus gene which gene or fragment encodes an antigen, wherein the poxvirus genome does not comprise a functional gene encoding a 3β-hydroxysteroid dehydrogenase/Δ⁵-Δ⁴isomerase, with the proviso that the non-poxvirus gene or fragment of a non-poxvirus gene is not a gene encoding varicella-zoster virus glycoprotein E, hepatitis B virus preS2-S protein or E. coli guanine phosphoribosyl transferase.

For the preparation of a vaccine, the poxvirus according to the invention is typically provided in a physiologically acceptable form. A person skilled in the art will be familiar with suitable poxvirus vaccine formulations given the large body of knowledge that was built up in the many years of use of VV in the vaccination against smallpox. For example, an appropriate number of particles of the recombinant poxvirus, e.g. 10⁴ to 10⁹ particles, are freeze dried in an appropriate volume, e.g. approximately 100 μl, of phosphate-buffered saline (PBS) in the presence of peptone and human albumin in, for example, a vial or an ampoule, preferably a glass ampoule. The lyophilisate can contain extenders, for example mannitol, dextran, sugar, glycine, lactose or polyviylpyrrolidone, or other excipients, for example antioxidants or stabilisers, suitable for parenteral administration. The vial or ampoule may then be sealed and may be stored for several months, preferably at a temperature below −20° C.

For vaccination, the lyophilisate may, for example, be made up to 0.1 to 0.2 ml of aqueous solution, preferably with physiological saline, and administered parenterally, for example by intradermal inoculation or by dermal abrasion. The vaccine of the invention may be infected intracutaneously. The mode of administration, the dose and the number of administrations can be optimised by those skilled in the art in a conventional manner. A high degree of immunity against the antigen is obtained by administration of the vaccine several times over a lengthy period of time.

Biological Significance of A44L Protein in Cellular Immunity

The present invention provides a poxvirus lacking a functional gene that encodes a 3β-HSD. An example of such a gene is the VV A44L gene. It has been found by the present applicants that A44L is an important virulence factor of VV and has potent immunosuppressive properties. The absence of a functional A44L gene therefore results in the immune response to the virus being enhanced. Investigations into the effects of a functional A44L gene and the effects of the absence thereof are described below.

3β-HSD Activity of VV Lacking A44L and Virulence Thereof

3β-HSD Activity

Expression of 3β-HSD activity in infected cells by strains of VV was measured in vitro by the conversion of [³H]-pregnenolone to [³H]-progesterone. As described in further detail in the Examples section below, all normal parental VV strains investigated exhibited 3β-HSD activity indicating v3β-HSD is highly conserved in VVs. The activity was not observed in strains of VV lacking a functional A44L gene. Upon infection of host cells, a VV without the A44b gene was found to generate a similar amount of infectious progeny virus as a VV with A44L. It appears unlikely, therefore, that the different levels of 3β-HSD activity seen reflect differences in the amount of virus present.

Effect of deletion of A44L on VV In Vivo

The role of the A44L protein in VV infection may be explored using various models of infection. The present inventors used a murine intranasal model. In the model, mice are infected with VV and then weighed individually and assessed for general signs of illness daily over a period of approximately 2 weeks.

Using such a method, the present inventors found that the infection phenotype is attenuated in mice infected with VV lacking A44L relative to parental and revertant viruses. Mice infected with parental or revertant VV generally lost over 20% of body weight during the infection and displayed severe signs of illness manifested by ruffled fur, reduced mobility and tachypnea. In contrast, mice infected with VV lacking A44L lost significantly less weight, recovered more rapidly and displayed very mild clinical signs of illness.

A further measure of infection progression is the change in body temperature with time after infection. To determine if A44L could affect body temperature during VV infection, rectal temperatures in mice were measured on a daily basis following infection with VV. No differences were seen in the early body temperatures (days 1 to 5 post infection (p.i.)) recorded in animals infected with VV lacking A44L and in animals infected with parental VV.

The B15R ORF of VV strain WR encodes a soluble IL-1β receptor and it was reported by Alcamí and Smith (Alcamí and Smith, 1992) that deletion of B15R results in a more virulent infection following intranasal inoculation of mice. Animals infected with VV lacking B15R displayed elevated temperatures at early times of infection suggesting that the B15R protein blocks IL-1β-induced fever (Alcamí and Smith, 1996). The fact that no such temperature effect was seen in the case of infection with VV lacking A44L indicates that A44L affects virulence through an alternative mechanism(s).

Previously, it had been reported that mice infected intranasally with vΔA44L (a recombinant VV WR strain lacking A44L but not comprising an expressed non-poxvirus gene or an expressed fragment of a non-poxvirus gene which gene or fragment encodes an antigen showed reduced mortality and relatively mild weight loss compared to those infected with parental strain WR (Moore and Smith, 1992). In a follow up study, Sroller et al. (Sroller et al., 1998) reported that loss of A44L had only a relatively modest effect on virulence following intranasal infection of mice with VV strain WR. They reported observing that only moderate attenuation of virulence was achieved by deletion of A44L and that immunogenicity of the VV was not affected. Deletion of the v3β-HSD from VV did not enhance antibody responses to infection and it was concluded that a role for A44L in immunosuppression was unlikely.

Despite the results reported by Sroller et al., the present inventors continued to investigate the immune response to VV strain WR lacking a functional A44L gene. The present inventors have found that the cell-mediated immune response is influenced by the presence or absence of A44L. The cell-mediated immune response is essential if effective immunity is to be achieved against certain types of pathogen.

A44L is non-essential for virus replication in vitro. However it appears to be well conserved amongst strains of VV and some other members of the poxvirus family. All VV strains tested expressed 3β-HSD activity and similar activity has been described in cells infected with VV strains Praha, LIVP and MVA (Sroller et al., 1998) as well as the avipoxviruses fowlpox and canarypox (Skinner et al., 1994) and a fish iridiovirus (Baker and Blasco, 1992). The gene is also highly conserved in several other sequenced orthopoxviruses (camelpox, ectromelia, cowpox and monkeypox) and other poxvirus genera (suipoxvirus, molluscipoxvirus and yatapoxvirus) suggesting the v3β-HSD plays an important role in poxvirus biology, perhaps in antiviral host defence. Interestingly, despite the presence of an A44L-like gene in several variola strains, in each case the gene has been disrupted by mutation and is not predicted to encode an active A44L counterpart (Aguado et al., 1992; Massung et al., 1993; Massung et al., 1994; Shchelkunov et al., 1995; Shchelkunov et al., 2000).

Clearance of VV Lacking A44L

Spread of VV in the Lung

In the intransal model for VV infection, the lung is the site of primary infection. As described in further detail in the Example section below, VV with or without A44L was found to establish infections in the lung and produce similar virus titres at 1 and 3 days p.i., indicating that A44L is not critical for the initial rounds of replication in the lung. However, by the time peak titres of virus occur, typically at 7 days p.i., the level of infectivity in the lungs was significantly greater in mice infected with parental VV than those infected with VV lacking A44L. By day 12, infectious virus was only detectable in a minority of animals infected with VV lacking A44L compared with all, or a substantial majority, animals infected with parental VV.

Spread in Cells Other than Lung Cells

To examine the ability of VVs to spread and replicate in extrapulmonary sites, homogenates of brain, spleen and liver of infected mice were assayed for infectious virus. In infected mice, virus titres were found to be below detection levels in all organs at day 1 p.i., however by day 3 p.i. infectious virus was recovered from the spleen and livers of all mice infected with W with or without A44L. By day 3 p.i. infectious virus was recovered from the brains of most of the mice infected with VV with A44L but from only a minority of mice infected with VV lacking A44L.

By day 7, virus titres were reduced in all of the organs examined from mice infected with VV lacking A44L, indicating that while this virus is capable of in vivo spread, it is cleared more rapidly from the brain, liver, spleen and lungs compared to parental VV.

To investigate the replication of VV in murine cells in vitro, cultures of primary fibroblasts may be prepared from the kidneys of naive BALB/c mice. The replication kinetics were analysed according to single-step (10 PFU/cell) or multistep (0.01 PFU/cell) growth curves, the latter conditions being particularly sensitive to accentuating small differences in growth between viruses. VV with and without A44L were found to have similar replication kinetics.

In conclusion, it has also been found that although
(i) VV with or without A44L replicates to similar levels in primary murine fibroblasts,
(ii) virus titres in the lungs of mice infected with VV with or without A44L were equivalent on day 1 p.i., and
(iii) VV with or without A44L were capable of spread and replication in secondary organs (brain, liver, spleen), the virus lacking A44L was cleared more rapidly. These findings indicate that A44L is not critical for the initial rounds of virus replication in the host and that the difference between the viruses is likely to be due to host antiviral mechanisms rather than altered replicative ability.
   Inflammatory Response to VV Lacking A44L

The observation that mice infected with VV lacking A44L were able to clear virus rapidly suggested a more effective antiviral host response against that virus. Therefore, the cellular inflammatory response to infection with VV in the lung was assessed.

BAL Cells from Lungs of VV-Infected Mice

BALs were performed as described in the Examples section below. Only few cells were recovered from BAL of animals 1 day post infection (p.i.) with VV, indicating there were only low levels of recruitment to the lung in the early phase of VV infection. BAL cell numbers increased over time with peak numbers 12 days p.i. for VV with A44L, and 7 days p.i. for VV lacking A44L. Significantly more cells were recovered at day 7 p.i. for mice infected with VV lacking A44L compared with mice infected with VV with A44L.

BAL cells were centrifuged onto slides for microscopic examination and differential cell counts were carried out. Uninfected mouse BAL cells were composed almost entirely of macrophages, with some (<10%) lymphocytes noted. The majority of inflammatory cells recruited to the lung during the course of VV infection were macrophages and lymphocytes, with granulocytes representing less than 5% of total cells at all time points tested.

Recruitment of lymphocytes to the lungs at day 7 was found to be statistically enhanced in mice infected with VV lacking A44L. That observation correlates with the more rapid clearance of that virus from the lungs of infected animals. Notably, for mice infected with VV lacking A44L by day 12 after infection the levels of total cells, macrophages and lymphocytes in BALs were all lower than on day 7, whereas for mice infected with VV containing A44L the cell numbers were higher on day 12 than on day 7.

A striking feature of the cellular inflammatory response to infection with VV lacking A44L was the early recruitment of lymphocytes to the lung. Flow cytometric analysis was used to examine lymphocyte subsets in BAL at day 7 p.i. A greater percentage of both CD4⁺ and CD8⁺ lymphocytes were observed in BAL from mice infected with VV lacking A44L compared to animals infected with VV with A44L. Less than 5% of BAL cells were DX5⁺ natural killer (NK) cells or B220⁺ B lymphocytes at this time. By day 7 p.i. with VV lacking A44L more cells were recruited to the lungs and a higher proportion of those were CD4⁺ and CD8⁺ T cells.

IFN-γ Production in Lungs of VV-Infected Mice

The results discussed indicate an increased presence of CD4⁺ and CD8⁺ T cells within the lungs of mice infected with VV lacking A44L.

IFN-γ plays an important role in antiviral host defence following infection of mice with VV or other poxviruses (Dalton et al., 1993; Huang et al., 1993; Muller et al., 1994). Given the marked enhancement in T cell recruitment to the lungs of mice infected with VV lacking A44L and the ability of both CD4⁺ and CD8⁺ T lymphocytes to produce antiviral cytokines, the levels of IFN-γ in BAL fluids from VV-infected mice were determined.

Low levels of IFN-γ were detected in lavage fluids 3 days p.i. and those increased markedly at days 7 and 10 p.i. Significantly higher levels of IFN-γ were detected in lavage fluids from mice infected with VV lacking A44L compared to animals infected with control parental viruses at day 7 p.i. IFN-γ levels at day 10 were also generally higher in mice infected with VV lacking A44L. Levels of IL-10 or IL-4 in BAL fluid were below the detection limit of their respective ELISA assays at each time points tested.

IFN-γ production was also determined after culturing lung cell suspensions in the presence of PMA and ionomycin. It was found that IFN-γ levels were very low in lung cell supernatants from mock-infected mice. A modest increase was observed in supernatants from lung cells 3 days p.i., and levels were elevated further from lung cells harvested day 7 and day 10 p.i. Consistent with IFN-γ levels observed in BAL, PMA-stimulated lung cells from mice infected with VV lacking A44L produced significantly more IFN-γ on day 7 and 10 than those from animals infected with VV with A44L p.i.

The elevated IFN-γ production detected in BAL fluids and lung cell suspensions from vΔA44L-infected mice suggests that the additional lymphocytes recruited to the lungs of these animals were producing IFN-γ. Intracellular staining of lung lymphocytes for IFN-γ on days 3, 7 and 10 p.i. showed that the proportion of both CD4⁺ and CD8+ lymphocytes positive for IFN-γ was higher at days 7 and 10 p.i. in the lungs of mice infected with VV lacking A44L.

Only a very low proportion of CD4⁺ or CD8⁺ cells (<5%) stained for IL-4 or IL-10.

Cytolytic Activity of Lymphocytes Isolated from VV-Infected Lungs

To assess the effect of VV A44L on virus-specific CTL activity, the cytotoxic activity of effector cells from lung cell suspensions may be examined. At day 5, primary CTL activity was very low in lung cell suspensions from all infected animals, however by day 7 significant CTL activity was detected against virus-infected targets.

The cytolytic activity of lung lymphocytes of mice infected with VV lacking A44L was greater than from mice infected with VV with A44L, and this trend was also observed using lung cells from animals 10 days p.i.

The increase in cytotoxic activity of lung cells from vΔA44L-infected mice may represent increased recruitment of CTL effectors to the lung or an increased activation state of the lung cells present.

To assess whether the CD8⁺ T lymphocytes were mediating CTL activity, day 7 lung cells from VV-infected mice were treated with complement and a mAb to murine CD8. The treatment abrogated virtually all CTL activity.

The numbers of CD8⁺ lymphocytes in the lung cell suspensions were determined by flow cytometry, and used to compare CTL activity between lung cells based on a CD8+ lung cell:target ratio. The enhanced lysis observed using lung cells from mice infected with VV lacking A44L is partly explained by the higher relative numbers of CD8⁺ T cells in this compartment. However, CTL from mice infected with VV lacking A44L also show an enhanced level of lysis on a per cell basis consistent with an enhanced activation state of virus-specific CTL within the T cell population.

Levels of Corticosterone in Plasma and Lungs of VV-Infected Mice

It has been found by the present inventors that VV lacking A44L is markedly attenuated in the murine intranasal model, and that this is accompanied by a more vigorous cellular inflammatory response in the lungs of infected mice. The A44L ORF encodes an active v3β-HSD enzyme capable of converting pregnenolone to progesterone (Moore and Smith, 1992) as discussed in further detail in the introduction. As there is growing evidence that GCs and other steroids may possess important immunomodulatory functions, the levels of corticosterone were determined in the plasma and lungs of VV-infected mice. Infection with VV induced an increase in corticosterone levels in plasma and lung extracts as early as 1 day p.i., and the levels increased further up to day 4. Levels were significantly lower in samples from mice infected with VV lacking A44L relative to mice infected with VV with A44L.

Corticosterone is released as part of the acute phase response to infection and other inflammatory trauma. Viral titres in the lungs were not significantly different between mice infected with VV with or without A44L at days 1, 2 and 4 p.i., nor were there differences in visible signs of illness or weight loss at these early time points. The enhanced levels of endogenous GCs observed in mice infected with VV containing A44L suggest that A44L may have a direct effect upon local and systemic steroid levels during VV infection.

Conclusions

The rapid clearance of vΔA44L coincided with an early influx of inflammatory leukocytes, in particular T lymphocytes, into the lung of mice infected with VV lacking A44L.

Functional analysis suggests that IFN-γ production and virus-specific CTL activity are enhanced in lymphocytes present in the lungs of mice infected with VV lacking A44L. That phenotype is consistent with the hypothesis that the A44L protein interferes with aspects of the pro-inflammatory host response.

Previously, the A44L gene from VV strain WR was shown to encode an active 3β-HSD enzyme (Moore and Smith, 1992). In view of the prior art information available regarding 3β-HSD enzymes (see above) a possible role for A44L in VV pathogenesis is in the production of steroid hormones such as GCs.

Previous studies have shown that restraint stress (RST) reduced the accumulation of leukocytes in the lungs of influenza virus-infected mice and that this response was dependent upon elevated serum GCs as treatment with the GC receptor antagonist RU486 restored cellular infiltration (Hermann et al., 1995). RST suppression of influenza virus-specific production of cytokines such as IL-2 and IFN-γ was also reversed by RU486 treatment (Dobbs et al., 1996). By analogy, the present findings that infection of mice with VV lacking A44L was associated with enhanced leukocyte infiltration and IFN-γ production in the lung is consistent with A44L-mediated enhancement of GC levels in vivo following infection with parental VV.

The increased levels of corticosterone in plasma and in the lung compared to uninfected mice and the significant increase in plasma and lung corticosterone levels in mice infected with VV with A44L compared to mice infected with VV lacking A44L are consistent with a direct effect of the A44L protein on increasing steroid levels in vivo. Alternatively, the differences observed might reflect the more severe illness caused by the VV with A44L as corticosterone is part of the acute phase response observed after infection or other inflammatory trauma. It is worth noting, however, that there were no differences in signs of illness, nor in virus titres recovered from the lungs of mice infected with VV with or without A44L at days 1, 2 and 4 p.i. while corticosterone levels differed at those times.

The mechanisms governing the immunomodulatory properties of the A44L protein in VV pathogenesis in vivo have not yet been established. Biochemically, mammalian 3β-HSD functions at multiple steps in steroid biosynthesis and the A44L protein has been shown previously to be capable of converting at least two substrates, pregnenolone and dehydroepiandrosterone (DHEA) to their respective 5-ketosteriods. The nature of the substrates available and the types and levels of different steroids produced in the lung following VV infection may provide important information as to the principal reaction/s catalysed in vivo by A44L.

A striking feature of the inflammatory response to VV lacking A44L is the rapid recruitment of leukocytes to the lungs of virus-infected mice, in particular CD4⁺ and CD8⁺ T lymphocytes with the potential to produce IFN-γ and CD8+ CTLs, indicating that A44L can suppress at least two important components of the antiviral host response.

Major histocompatability complex (MHC) class I-restricted, CD8⁺ CTL and the antiviral cytokine IFN-γ are believed to play an important role in the resolution of acute infection by poxviruses such as VV (Huang et al., 1993; Ramsay et al., 1993; Ruby and Ramshaw, 1991). VV is known to be very sensitive to IFN-γ in vitro (Melkova and Esteban, 1994) and IFN-γ production is critical for recovery of mice after VV infection (Dalton et al., 1993; Huang et al., 1993; Karupiah et al., 1990; Ruby and Ramshaw, 1991). Poxviruses encode a number of proteins that are expressed intracellularly and extracellularly to interfere with the antiviral effects of IFN-γ. However, a viral IFN-γ receptor secreted from VV-infected cells does not neutralize mouse IFN-γ (Alcamí and Smith, 1995; Mossman et al., 1995). CD8⁺ CTLs are key mediators of viral clearance by cytolysis of virus-infected cells and the secretion of cytokines such as IFN-γ and TNF-α. Interestingly, GCs have also been reported to suppress CTL activity (Schleimer et al., 1984).

It is apparent from the inventors' data presented above that A44L is immunosuppressive and interferes with the early events in the host cellular response to VV infection, thus contributing to protracted virus replication and improved virus dissemination within the host. It follows that the removal of the A44L gene from poxyvirus vaccines, for example modified virus Ankara (MVA), enhances vaccine immunogenicity, particularly T cell responses to infection.

### EXAMPLES

Cells and Viruses

BS-C-1 (African green monkey, epithelial), CV-1 (African green monkey, fibroblast) and TK-143 (human osteosarcoma) cells were gown in Dulbecco's modified Eagle's medium (DMEM) supplemented with 10% heat-inactivated foetal bovine serum (FBS). The origins of the VV strains used herein were as described by Alcamí et al. (Alcamí et al., 1998). VV strain WR was grown and partially purified through sucrose cushions as described by Mackett et al. (Mackett et al., 1985). VV strain WR is available from ATCC under number VR-119.

Construction of Recombinant Viruses

Wild-type VV strain WR is referred to hereinafter as vA44L in view of its having a full viral A44L gene. Recombinant virus vΔA44L, in which 86% of the A44L ORF of VV strain WR was replaced with the selectable marker E. coli guanine phosphoribosyl transferase (Ecogpt), was constructed as described by Moore and Smith (Moore and Smith, 1992).

A revertant virus, based on vΔA44L and into which a functional A44L gene had been inserted was used as a control to ensure that any phenotypic differences of vΔA44L were due to loss of the A44L protein and not due to mutations elsewhere in the genome.

Revertant virus vA44L-rev was constructed by replacing the Ecogpt of vΔA44L with the A44L gene. Plasmid pJM5 was constructed by cloning a 2318-bp HincII fragment of VV strain WR containing the A44L ORF and flanking regions into the unique SmaI site of pUC119. Plasmid pJM5 was transfected into vΔA44L-infected cells and mycophenolic acid (MPA)-resistant recombinant viruses were isolated as described by Falkner and Moss (Falkner and Moss, 1990). These were grown on hypoxanthine guanine phosphoribosyl-transferase negative D980R cells in the presence of 6-thioguanine and plaque isolates corresponding to deletion mutant or revertant viruses (A44L-rev) were identified.

Analysis of vA44L, vΔA44L and vA44L-rev viruses by PCR confirmed that the entire A44L gene was present in only vA44L and vA44L-rev viruses and that the genes flanking the A44L locus were similar in each of the three viruses. Southern blots of the DNA of the three viruses showed that the genomes were as predicted. The plaque morphology formed on BS-C-1 cells by vA44L, vΔA44L and vA44L-rev viruses was indistinguishable, confirming that loss of A44L does not affect virus replication in vitro.

3β-HSD Assay

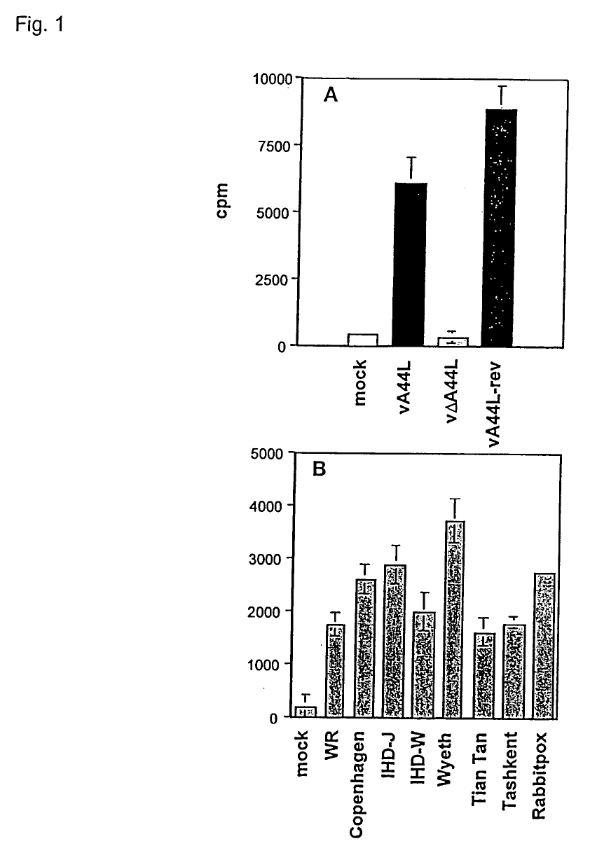

Monolayers of CV-1 cells in 24-well plates were mock-infected or infected in triplicate with VV at 10 PFU/cell. At 10 h p.i. 3β-HSD activity was measured by conversion of [³H]-pregnenolone to [³H]-progesterone as described by Moore and Smith (Moore and Smith, 1992). All infections were performed in triplicate and results are expressed as mean±SEM. The background values from non-enzymatic conversion to progesterone (measured in ethanol-fixed monolayers) were subtracted from each value. The data for vA44L, vΔA44L or A44L-rev are shown in FIG. 1A. As seen in that Figure, vA44L- and vA44L-rev-infected CV-1 cells produced 3β-HSD activity but mock- and vΔA44L-infected cells did not.

To extend these findings, a range of VV strains were examined for their ability to produce 3β-HSD activity in vitro. Eight strains of VV were assayed for 3β-HSD activity at 8-10 h p.i. The data for the eight different VV strains are shown in FIG. 1B. All eight strains tested were able to convert [³H]-pregnenolone to [³H]-progesterone, indicating v3β-HSD is highly conserved in VVs.

Cells from duplicate wells were harvested during the experiment and the amount of infectious virus was found to be similar in all samples, indicating it is unlikely that the different levels of 3β-HSD activity seen reflect differences in the amount of virus present.

Assay for Virus Virulence

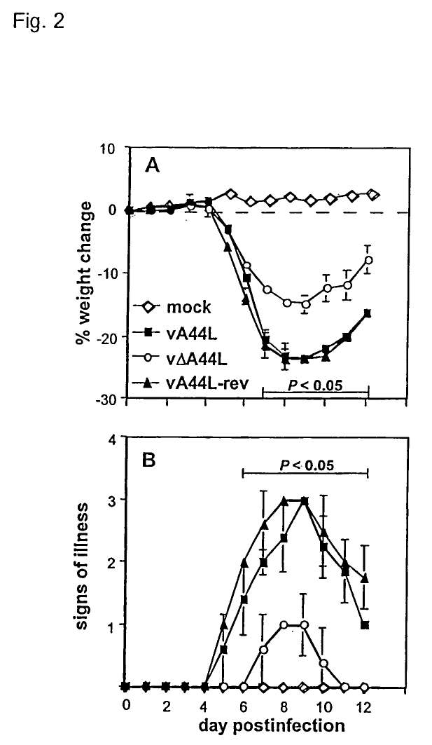

The role of the A44L protein in VV infection was explored using a murine intranasal model of infection. Groups of five 6 to 8 week-old female BALB/c mice infected with 10⁴ PFU of vA44L, vΔA44L or vA44L-rev. Mice were subjected to brief anesthesia and were inoculated intranasally with 10⁴ or 10⁵ PFU of VV in PBS on day 0. Each day, mice were weighed individually and the results are expressed as the mean percentage weight change of each group±SEM compared with the weight immediately prior to infection. Mice were also monitored for signs of illness, and they were scored from 1 to 4. Data from each day are expressed as the mean±SEM from 5 mice. Mice suffering a severe infection or having lost >25% of their original body weight were sacrificed. The change in weight and the signs of illness seen in the mice in the days immediately after infection are shown in FIGS. 2A and 2B in which ⋄=mock-infected, ▪=infected with vA44L, ◯=infected with vΔA44L and A=infected with A44L-rev.

It is seen in FIG. 2 that the infection phenotype is attenuated in vΔA44L relative to both vA44L and vA44L-rev viruses. Mice infected with vA44L or vA44L-rev lost over 20% of body weight during the infection (FIG. 2A) and displayed severe signs of illness manifested by ruffled fur, reduced mobility and tachypnea (FIG. 2B). In contrast, mice infected with vΔA44L lost significantly less weight, recovered more rapidly and displayed very mild clinical signs of illness. There was no significant difference between the weight loss profiles of mice infected with vA44L or vA44L-rev indicating that the attenuation seen with vΔA44L is due to loss of the A44L gene.

P values were determined using the Student's t-test and indicate the mean % weight changes or signs of illness of mice infected with vΔA44L that were significantly different from both those of mice infected with vA44L or A44L-rev.

Spread of VV in the Lung, Brain, Spleen and Liver

WR is a neurovirulent strain of VV that was derived by repeated passage in suckling mouse brain (Bronson and Parker, 1941). In the murine intranasal model, VV infection is accompanied by extensive respiratory infection and virus dissemination to multiple organs (Turner, 1967; Williamson et al., 1990). In the intranasal model for VV infection, the lung is the site of primary infection. To examine the ability of the three viruses to spread and replicate in extrapulmonary sites, homogenates of brain, spleen and liver were also assayed for infectious virus.

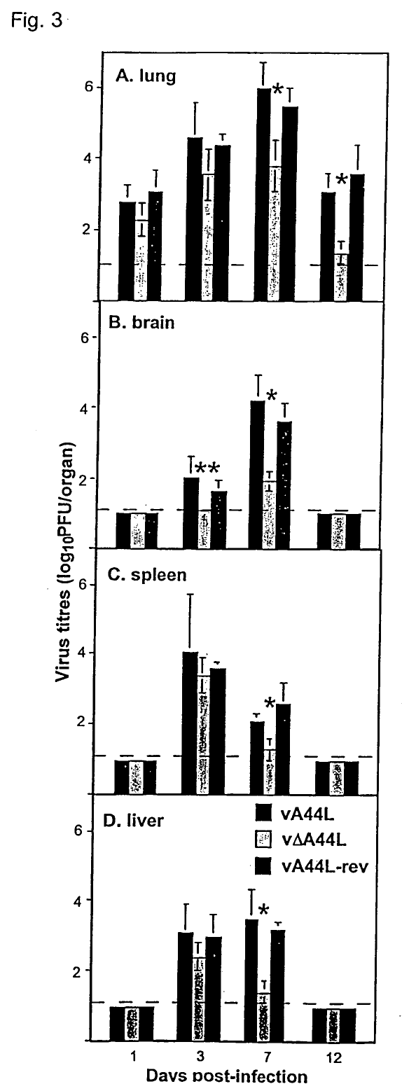

Groups of 5 mice were infected intranasally with 10⁴ PFU of vA44L, vΔA44L or vA44L-rev and the lungs, brains, spleens and livers were harvested at noted times p.i. After sacrifice of the mice, their lungs, brains, livers and spleens were removed, dounce homogenized, frozen and thawed three times and sonicated and stored at −70° C. The titre of infectious virus was determined by plaque assay on BS-C-1 cells. The titres of vA44L, vΔA44L and A44L-rev in the lungs (A), brains (B), spleens (C) and livers (D) are shown in FIG. 3. Virus titres are expressed as mean log₁₀PFU per organ, with SEM. The broken line indicates the minimum detection limit of the plaque assay. Columns marked with an asterisk represent virus titres from vΔA44L-infected mice that were significantly different to those from both vA44L- and vA44L-infected animals. *, P<0.05, **, P<0.02.

As seen in FIG. 3A, the three viruses established infections in the lung and produced similar virus titres at 1 and 3 days p.i., indicating that A44L is not critical for the initial rounds of replication in the lung. However, by the time peak titres of virus occurred at 7 days p.i. the level of infectivity in the lungs of vA44L and vA44L-rev-infected mice was significantly greater than that in mice infected with vΔA44L. By day 12, infectious virus could be detected in the lungs of only 1/5 vΔA44L-infected animals compared to 5/5 in each of the other groups.

The results of the brain, spleen and liver experiments are shown in FIGS. 3B, C and D respectively. Virus titres were below detection levels in all organs at day 1 p.i., however by day 3 p.i. infectious virus was recovered from brain, spleen and liver of all mice infected with vA44L or vA44L-rev, and from the spleen and livers of all vΔA44L-infected animals. Infectivity titres were similar in all groups at this time, except in the brain of vΔA44L-infected mice, where virus was recovered from only 1/5 animals. By day 7, virus titres were reduced in all of the organs examined from vΔA44L-infected mice, indicating that while this virus is capable of in vivo spread, it is cleared more rapidly from the brain, liver, spleen and lungs compared to vA44L and vA44L-rev viruses. Virus was still present in lungs of VV-infected animals 12 days p.i., however it had been cleared from all other organs examined by this time.

To investigate if vA44L, vΔA44L and vA44L-rev could replicate to similar levels in murine cells in vitro, cultures of primary fibroblasts were prepared from the kidneys of naive BALB/c mice. The replication kinetics of the three viruses were similar during single-step (10 PFU/cell) or multistep (0.01 PFU/cell) growth curves; the latter conditions are particularly sensitive to accentuating small differences in growth between viruses.

Recovery of Bronchoalveolar Lavage (BAL) and Lung Cells

Mice were infected with 10⁴ PFU of vA44L, vΔA44L or vA44L-rev. BAL fluid was obtained from mock- and VV-infected mice at various times p.i. Mice were sacrificed and the lungs of each mouse were inflated five times with a 1 ml volume of PBS containing 10 U/ml of heparin through a blunted 23-guage needle inserted into the trachea. BAL was centrifuged at 3,000 rpm for 10 min and the supernatant was removed and frozen at −20° C. for analysis of cytokines by ELISA. BAL cells were treated with Tris-NH₄Cl (0.14 M NH₄Cl in 17 mM Tris, adjusted to pH 7.2) to lyse erythrocytes, washed twice and resuspended in cold RPMI 1640 medium supplemented with 10% FBS. BAL cells from individual mice were cytocentrifuged onto glass slides, air dried and stained with haematoxylin and eosin for differential cell counts. Lungs were removed from mock- and VV-infected mice and single cell suspensions were prepared by sieving through a 100-μm nylon mesh followed by hypotonic lysis of erythrocytes. Cell viability in all samples was assessed using trypan blue exclusion.

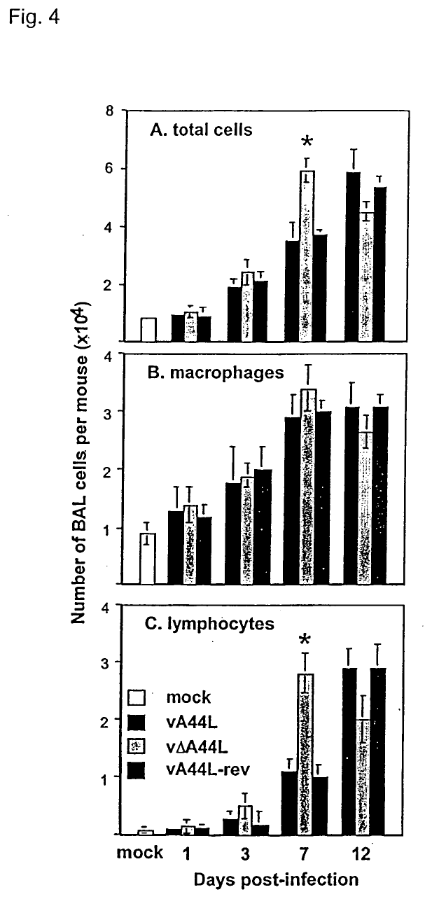

BAL cells were counted to determine the numbers of (A) total cells, (B) macrophages and (C) lymphocytes from mock-infected and VV-infected mice. The results are shown in FIG. 4 in which columns represent the mean cell yield per mouse±SEM from groups of 4-5 mice. Columns marked with an asterisk represent mean cell numbers recovered from vΔA44L-infected mice that were significantly different (*, P<0.05) to those of both vA44L and vA44L-rev-infected mice.

Only few cells were recovered from BAL of mock-infected animals 1 day p.i. with VV, indicating there were only low levels of recruitment to the lung in the early phase of VV infection (FIG. 4A). BAL cell numbers increased over time with peak numbers 12 days p.i. for vA44L or vA44L-rev, and 7 days after infection for vΔA44L. Significantly more cells were recovered from vΔA44L-infected mice at day 7 p.i. compared to other VV-infected groups.

Cytospins of BAL cells were prepared and stained with haematoxylin and eosin for differential cell counts and the results are shown in FIGS. 4B and 4C. Uninfected mouse BAL cells were composed almost entirely of macrophages, with some (<10%) lymphocytes noted. The majority of inflammatory cells recruited to the lung during the course of VV infection were macrophages (FIG. 4B) and lymphocytes (FIG. 4C), with granulocytes representing less than 5% of total cells at all time points tested. By day 12 after vΔA44L infection the levels of total cells, macrophages and lymphocytes in BALs were all lower than on day 7, whereas with vA44L and vA44L-rev the cell numbers were higher on day 12 than day 7.

Flow Cytometric Analysis of Cell Surface and Intracellular Antigens

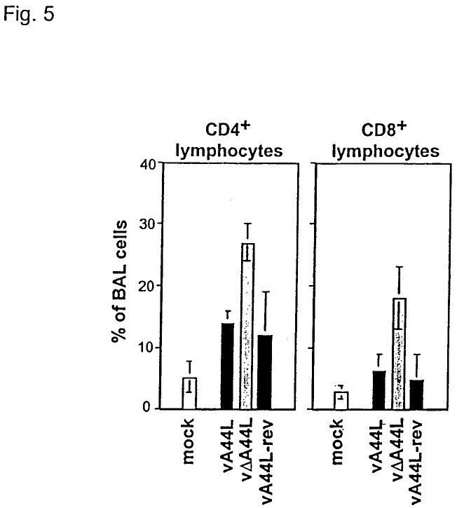

Lymphocytes from BAL of VV-infected mice were analysed. Groups of 5 BALB/c mice were mock-infected or infected with 10⁴ PFU of vA44L, vΔA44L, or A44L-rev. At 7 days p.i., BAL cells were recovered, and blocked with 10% normal rat serum and 0.5 μg of Fc block (Pharmingen) in FACs buffer (PBS containing 0.1% bovine serum albumin and 0.1% sodium azide) on ice for 20 min. Cells were stained with fluorescein isothiocynated (FITC) anti-CD4, Tri-colour anti-CD8 and phycoerythrin (PE)-labelled anti-CD3 or isotype antibody controls (all from Caltag, Burlingame, Calif.). Lymphocytes were identified by their characteristic FSC/SSC profile and by expression of CD3. The distribution of cell surface markers was determined on a FACScan flow cytometer with CellQUEST software (Becton Dickenson, Mountain View, Calif.). A lymphocyte gate was used to select at least 20,000 events. Data from the experiments are shown in FIG. 5. The data shown are the mean percentage of BAL cells±SEM from 4-5 individual mice, and are representative of two independent experiments.

The results of the flow cytometric analysis at day 7 p.i. is shown in FIG. 5. A greater percentage of both CD4⁺ and CD8⁺ lymphocytes was observed in BAL from vΔA44L-infected mice compared to animals infected with vA44L or vA44L-rev. Less than 5% of BAL cells were DX5+natural killer (NK) cells or B220⁺ B lymphocytes at this time. The data show that by day 7 p.i. with vΔA44L more cells were recruited to the lungs (FIG. 4) and a higher proportion of those were CD4⁺ and CD8⁺ T cells.

To detect intracellular cytokines, 10⁶ lung cells/ml were stimulated with 50 ng/ml PMA (Sigma), 500 ng/ml ionomycin (Calbiochem) in the presence of 10 μg/ml brefeldin A (Sigma) for 5 h at 37° C. Cells were washed with FACs buffer and stained with Tri-colour anti-CD4 and FITC anti-CDB for 30 min on ice and then fixed for 30 min at room temperature with 2% paraformaldehyde in PBS. Samples were permeabilized with 0.5% saponin in FACS buffer for 10 min. PE-conjugated anti-mouse IFN-γ (clone XMG1.2 Pharmingen) was added for a further 30 min at room temperature and the cells were washed once with 0.5% saponin in FACs buffer and twice in FACs buffer alone. Cells were analyzed on a Becton Dickinson flow cytometer collecting data on at least 20,000 lymphocytes. Intracellular staining of lung lymphocytes for IFN-γ on days 3, 7 and 10 p.i. showed that the proportion of both CD4⁺ and CD8⁺ lymphocytes positive for IFN-γ was higher at days 7 and 10 p.i. in the lungs of mice infected with VV lacking A44L.

ELISA for Cytokines

Groups of 4-6 BALB/c mice were mock-infected or infected with 10⁴ PFU of vA44L, vΔA44L, or vA44L-rev. At day 3, 7 and 10 mice were sacrificed, BALs were performed and single cell suspensions were prepared from lung tissue. BAL samples were centrifuged and the levels of IFN-γ present in the supernatant was determined by ELISA.

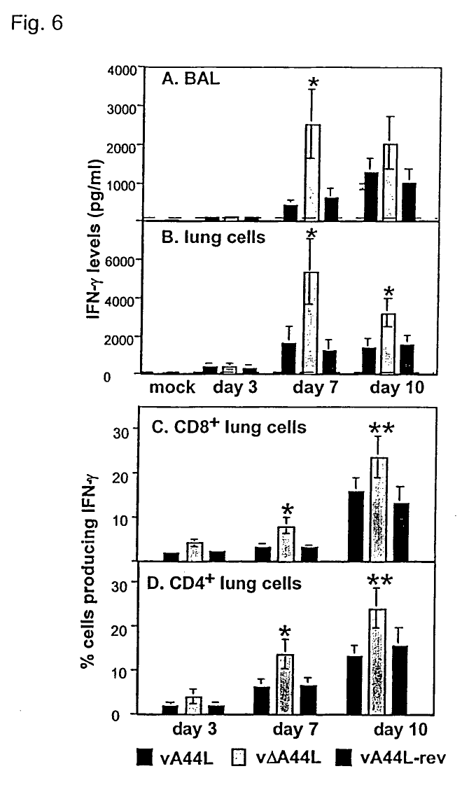

IFN-γ, IL-10 and IL-4 in BAL fluid and culture supernatants was quantified using OptEIA kits from Pharmingen according to the instructions provided. The concentration of cytokine in each sample was calculated from a standard curve and expressed as ng/ml. Levels of IFN-γ in BAL fluids of VV-infected mice are shown in FIG. 6A. Values represent the mean, with SEM, from 2 groups (n=3/group). The dashed line represents the detection limit of the IFN-γ ELISA (50 pg/ml).

As seen in FIG. 6A, IFN-γ levels in BAL fluids from mock-infected animals were below the detection limit of the assay. Low levels of IFN-γ were detected in lavage fluids 3 days p.i. and these increased markedly at days 7 and 10 p.i. Significantly higher levels of IFN-γ were detected in lavage fluids from vΔA44L-infected mice compared to animals infected with control viruses at day 7 p.i. IFN-γ levels at day 10 were also generally higher in mice infected with vΔA44L (FIG. 6A). Levels of IL-10 or IL-4 in BAL fluid were below the detection limit of their respective ELISA assays at each time point tested.

IFN-γ production was determined after culturing lung cell suspensions in the presence of PMA and ionomycin for 5 h at 37° C. Cells were pelleted and levels of IFN-γ present in the supernatant determined by ELISA. The data are shown in FIG. 6B. Values represent the mean±SEM, from two groups (n=3/group). The dashed line represents the detection limit of the IFN-γ ELISA (50 pg/ml). In the Figure, it is seen that IFN-γ levels were very low in lung cell supernatants from mock-infected mice. A modest increase was observed in supernatants from lung cells 3 days p.i., and levels were elevated further from lung cells harvested day 7 and day 10 p.i. Consistent with IFN-γ levels observed in BAL, PMA-stimulated lung cells from vΔA44L-infected mice produced significantly more IFN-γ than those from animals infected with vA44L or vA44L-rev on day 7 and 10 p.i. (FIG. 6B).

The elevated IFN-γ production detected in BAL fluids and lung cell suspensions from vΔA44L-infected mice suggested that the additional lymphocytes recruited to the lungs of these animals were producing IFN-γ. Intracellular staining of lung lymphocytes for IFN-γ on days 3, 7 and 10 p.i. demonstrated that the proportion of both CD4⁺ and CD8+ lymphocytes positive for IFN-γ was higher in the lungs of vΔA44L-infected mice at days 7 and 10 p.i. (FIG. 6C, D). Only a very low proportion of CD4⁺ or CD8⁺ cells (<S %) stained for IL-4 or IL-10 in any of the groups.

To assess intracellular production of IFN-γ by lung lymphocytes from mice 7 days after intranasal VV, lung cells were stimulated with PMA and ionomycin for 4 h, brefeldin A was added to retain cytokines in the cytoplasm. Cells were stained with FITC-labelled anti-CD8, APC-labelled anti-CD4, and after permeabilization using saponin, with PE-labelled anti-IFN-γ before analysis by three-color flow cytometry The data are shown in FIGS. 6C and 6D indicating the percentages of CD8⁺ (C) or CD4⁺ (D) T cells producing IFN-γ. Values are averaged from two groups (n=3/group). The frequency of IL-4-producing cells was below the detection limit (<2%) and is not shown. *, P<0.05, **, P<0.02.

Cytotoxic Assays

NK cell cytotoxicity and virus-specific CTL activity were analysed in effector cells from lung cell suspensions of VV-infected mice. Groups of 6 mice were infected with 10⁴ PFU of VV. At days 7 and 10 mice were sacrificed and lung cell suspensions were prepared.

NK cell cytotoxicity and virus-specific CTL activity in lung cell suspensions was assayed in a standard ⁵¹Cr-release assays. NK-mediated lysis was tested on YAC-1 cells while P815 cells (H-2^{d}, mastocytoma) were used as targets for virus-specific CTL lysis.

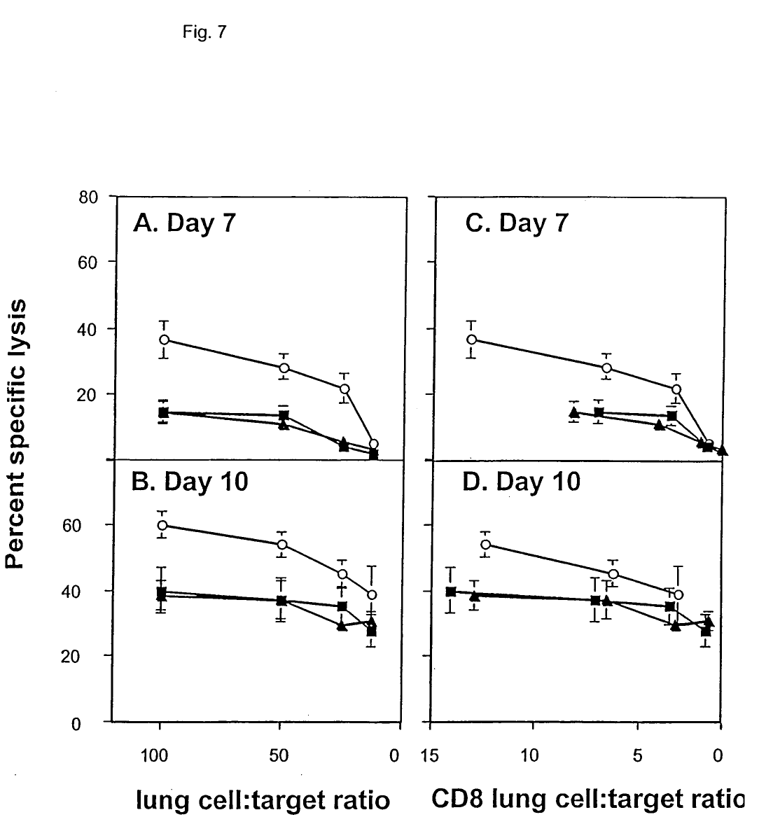

Prior to labelling with Na₂⁵¹CrO₄ (150 μCi per 3×10⁶ cells) P815 cells were mock-infected or infected with VV WR at 10 PFU/cell for 2 h at 37° C. Uninfected YAC-1 cells were labelled as above. Serial dilutions of effector cells were incubated in triplicate cultures with either uninfected or VV-infected target cells in 100 μl of RPMI 1640 supplemented with 10% FBS in 96-well V-bottomed plates at 37° C. in 5% CO₂. After 4 h (YAC-1) or 6 h (P815) cells were pelleted and 50 μl of the supernatant was transferred to a Lumaplate-96 (Packard Instrument Company Inc., USA) and counted using a Packard Microplate Scintillation counter. The percentage of specific ⁵¹Cr release was calculated as: % specific lysis=[(experimental release−spontaneous release)]/(total detergent release−spontaneous release)]×100%. The results of the assays are shown in FIG. 7. FIGS. 7A and C show data from mice sacrificed after 7 days, FIGS. 7B and D show data from mice sacrificed after 10 days. Data are shown for mice infected with vA44L (▪), vΔA44L (◯), or vA44L-rev (▴). Data are expressed as the mean percent specific lysis+SEM from two groups of 3 mice plotted against the lung cell:target ratio (A, B) or the CD8⁺ lung cell:target ratio (C, D). Lysis of uninfected P815 cells by day 7 and day 10 effector cell populations was always <10% at an effector:target ratio of 100:1.

In some experiments CD8⁺ cells were depleted from lung cell suspensions by incubation at 37° C. with an anti-CD8 mAb (clone 3.115 (Sarmiento et al., 1980)) in the presence of human complement. Analysis by flow cytometry demonstrated selective depletion of the CD8⁺ cell population. Depleted cells were added to cytotoxicity assays without adjustment for the depletion in cell number.

At day 5, primary CTL activity was very low in lung cell suspensions from all infected animals (<10% of specific cell lysis), however by day 7 significant CTL activity was detected against virus-infected targets (FIG. 7A). The cytolytic activity of lung lymphocytes from vΔA44L-infected mice was greater than from mice infected with vA44L or vA44L-rev, and this trend was also observed using lung cells from animals 10 days p.i. (FIG. 7B). All lung cell suspensions showed very weak cytotoxic activity against uninfected P815 cells (<10% of specific cell lysis at effector:target ratio of 100:1). Furthermore, no significant cytotoxic activity was observed against the NK-sensitive YAC-1 cell line.

The increase in cytotoxic activity of lung cells from vΔA44L-infected mice may represent increased recruitment of CTL effectors to the lung or an increased activation state of the lung cells present. To confirm that CD8⁺ T lymphocytes were mediating CTL activity in our system we treated day 7 lung cells from WV-infected mice with complement plus a mAb to murine CD8. This treatment abrogated virtually all CTL activity (undepleted=56% specific lysis, complement alone=46%, complement plus anti-CD8=3%, at effector:target ratio of 100:1).

The numbers of CD8⁺ lymphocytes in the lung cell suspensions were determined by flow cytometry, and used to compare CTL activity between lung cells based on a CD8⁺ lung cell:target ratio (FIG. 7C, D). The enhanced lysis observed using lung cells from vΔA44L-infected mice is partly explained by the higher relative numbers of CD8⁺ T cells in this compartment (7.4±2.2%, 13.6±2.0% and 8.0±1.9% of total lung cells at day 7, and 14.5±3.8%, 25.1±4.7% and 13.5±3.0% of total lung cells at day 10 from mice infected with vA44L, vΔA44L and vA44L-rev, respectively). However, CTL from vΔA44L-infected mice also show an enhanced level of lysis on a per cell basis consistent with an enhanced activation state of virus-specific CTL within the T cell population (FIG. 7C, D).

Corticosterone Levels in Plasma and Lung

Corticosterone levels in plasma and lungs were measured after intranasal infection with VV. Plasma and lung extracts were collected from BALB/c mice under low stress conditions after intranasal infection with 10⁵ PFU of vA44L, vΔA44L, or A44L-rev. To guard against fluctuations due to circadian rhythm, samples were obtained between 9.00 and 10.00 am each day of assessment. Mice were sacrificed by cervical dislocation and exsanguinated within 4 min of disturbance. Blood was collected in EDTA-coated tubes on ice and was then centrifuged at 3,000 rpm for 10 min. Plasma was collected and stored at −20° C. Lungs were removed immediately after exsanguination, washed once in PBS and placed on ice. Tissue was homogenised and extracted in 2 ml of methanol. Corticosterone levels in plasma and lung extracts were determined by radioimmunoassay using a rat corticosterone ³H kit (ICN Pharmaceuticals, Orangeburg, N.Y.). For lung extracts, excess methanol was evaporated and the dried pellet was resuspended in 0.5 ml of the steroid buffer supplied with the kit. Corticosterone levels were determined from individual mice using a standard curve and expressed as ng/ml for plasma or ng/g of lung tissue for lung extracts.

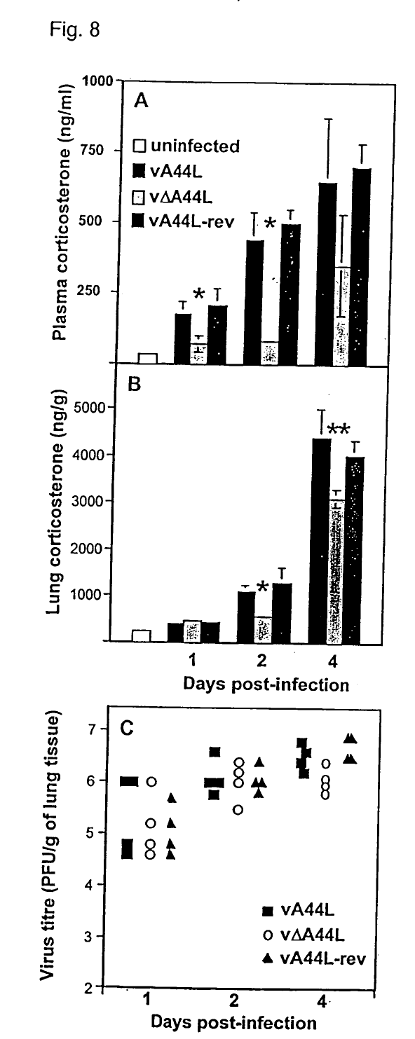

The results from the assays are shown in FIG. 8. Data represent mean±SEM of 4 or 5 mice per time point and are expressed as ng/ml of plasma or as ng/g of lung tissue.

Columns marked with an asterisk represent corticosterone levels from vΔA44L-infected mice that were significantly different to those from vA44L- and vA44L-rev-infected mice. *, P<0.05, **, P<0.02.

Titres of infectious virus in the lungs of mice after infection with los PFU of VV were also determined. Virus titres were determined by plaque assay on BS-C-1 cells and are expressed as PFU/g of lung tissue. Infection with VV induced an increase in corticosterone levels in plasma (FIG. 8A) and lung extracts (FIG. 8B) as early as 1 day p.i., and the levels increased further up to day 4. Levels were similar in vA44L and vA44L-rev-infected mice at all time points, but were significantly lower in plasma at day 1 and 2 and in lung extracts at day 2 and 4 from vΔA44L-infected mice, relative to control-infected animals. Corticosterone is released as part of the acute phase response to infection and other inflammatory trauma, however viral titres in the lungs were not significantly different between vA44L-, vΔA44L- or vA44L-rev-infected mice at days 1, 2 and 4 p.i. (FIG. 8C), nor were there differences in visible signs of illness or weight loss at these early time points at this dose or at 10⁴ PFU (FIG. 2). The enhanced levels of endogenous GCs observed in vA44L and vA44L-rev-infected mice suggest that A44L may have a direct effect upon local and systemic steroid levels during VV infection.

VV Expressing a Gene or Gene Fragment Encoding a Foreign Antigen but Lacking A44L

The gene encoding the antigen is introduced into the VV in a similar manner to that described above in relation to deletion of A44L. Cells are infected with VV and transfected with a plasmid recombination vector. The is plasmid contains a gene that encodes the antigen. The plasmid also comprises a correctly oriented VV promoter, a W early transcriptional termination sequence (TTTTTNT) flanked by two DNA sequences homologous to sequences of the VV that may be disrupted without compromising the viability of the VV.

Within the infected cells, homologous recombination between the VV genome and the plasmid DNA results in insertion of the foreign gene into the VV genome. The recombinant genome is replicated and packaged into infectious progeny virus. The progeny virus is screened for desired recombinants and insertion of the desired gene is confirmed by PCR.

VV Vaccine

10⁶ to 10⁷ infectious particles of the recombinant VV are freeze dried in 100 μl of PBS in the presence of 2% peptone and 1% human albumin in an ampoule, preferably a glass ampoule. The ampoule is then sealed and stored at a temperature below −20° C.

For vaccination, the lyophilisate is made up to 0.1 ml of aqueous solution with physiological saline. The vaccine is administered by intradermal inoculation.

### REFERENCES

Aguado, B., Selmes, I. P. and Smith, G. L. (1992) Nucleotide sequence of 21.8 kbp of variola major virus strain Harvey and comparison with vaccinia virus. J. Gen. Virol., 73, 2887-2902.
Alcamí, A. and Koszinowski, U. H. (2000) Viral mechanisms of immune evasion. Trends Microbiol., 8, 410-418.
Alcamí, A. and Smith, G. L. (1996) A mechanism for the inhibition of fever by a virus. Proc. Natl. Acad. Sci. USA, 93, 11029-11034.
Alcamí, A. and Smith, G. L. (1992) A soluble receptor for interleukin-1 beta encoded by vaccinia virus: a novel mechanism of virus modulation of the host response to infection. Cell, 71, 153-167.
Alcamí, A. and Smith, G. L. (1995) Vaccinia, cowpox, and camelpoxviruses encode soluble gamma interferon receptors with novel broad species specificity. J. Virol., 69, 4633-4639.
Alcamí, A., Symons, J. A., Collins, P. D., Williams, T. J. and Smith, G. L. (1998) Blockade of chemokine activity by a soluble chemokine binding protein from vaccinia virus. J. Immunol., 160, 624-633.
Ashwell, J. D., Lu, F. W. and Vacchio, M. S. (2000) Glucocorticoids in T cell development and function. Annu. Rev. Immunol., 18, 309-345.
Baker, M. E. and Blasco, R. (1992) Expansion of the mammalian 3 beta-hydroxysteroid dehydrogenase/plant dihydroflavonol reductase superfamily to include a bacterial cholesterol dehydrogenase, a bacterial UDP-galactose-4-epimerase, and open reading frames in vaccinia virus and fish lymphocystis disease virus. FEBS Lett. 301, 89-93.
Blasco, R., Cole, N. B. and Moss, B. (1991) Sequence analysis, expression, and deletion of a vaccinia virus gene encoding a homolog of profilin, a eukaryotic actin-binding protein. J. Virol., 65, 4598-4608.
Boumpas, D. T., Chrousos, G. P., Wilder, R. L., Cupps, T. R. and Balow, J. E. (1993) Glucocorticoid therapy for immune-mediated diseases: basic and clinical correlates. Ann. Intern. Med., 119, 1198-1208.
Boumpas, D. T., Paliogianni, F., Anastassiou, E. D. and Balow, J. E. (1991) Glucocorticosteroid action on the immune system: molecular and cellular aspects. Clin. Exp. Rheumatol., 9, 413-423.
Bronson, L. H. and Parker, R. F. (1941) The neutralization of vaccine virus by immune serum: titration by the intracerebral inoculation of mice. J. Bact., 41, 56-57.
Bugbee, L. M., Like, A. A. and Stewart, R. B. (1960) The effects of cortisone on intradermally induced vaccinia infection in rabbits. J. Infect. Dis., 106, 166-173.
Daigle, J. and Carr, D. J. (1998) Androstenediol antagonizes herpes simplex virus type 1-induced encephalitis through the augmentation of type I IFN production. J. Immunol., 160, 3060-3066.
Dalton, D. K., Pitts-Meek, S., Keshav, S., Figari, I. S., Bradley, A. and Stewart, T. A. (1993) Multiple defects of immune cell function in mice with disrupted interferon-gamma genes. Science, 259, 1739-1742.
Dhabhar, F. S., Miller, A. H., Stein, M., McEwen, B. S. and Spencer, R. L. (1994) Diurnal and acute stress-induced changes in distribution of peripheral blood leukocyte subpopulations. Brain. Behav. Immun., 8, 66-79.

Dobbs, C. M., Feng, N., Beck, F. M. and Sheridan, J. F. (1996) Neuroendocrine regulation of cytokine production during experimental influenza viral infection: effects of restraint stress-induced elevation in endogenous corticosterone J. Immunol., 157, 1870-1877
Dorrell, L., O'callaghan, C. A., Britton, W., Hambleton, S., McMichael, A., Smith G. L., Rowland-Jones, S and Blanchard, T. J. (2001) Recombinant modified vaccinia virus Ankara efficiently restimulates human cytotoxic T lymphocytes in vitro. Vaccine, 19, 327-336.
Falkner, F. G. and Moss, B. (1990) Transient dominant selection of recombinant vaccinia viruses. J. Virol., 64, 3108-3111.
Fong, Y., Tracey, K. J., Moldawer, L. L., Hesse, D. G., Manogue, K. B., Kenney, J. S., Lee, A. T., Kuo, G. C., Allison, A. C., Lowry, S. F. and et al. (1989) Antibodies to cachectin/tumor necrosis factor reduce interleukin 1 beta and interleukin 6 appearance during lethal bacteremia. J. Exp. Med., 170, 1627-1633.
Goebel, S. J., Johnson, G. P., Perkus, M. E., Davis, S. W., Winslow, J. P. and Paoletti, E. (1990) The complete DNA sequence of vaccinia virus. Virology, 179, 247-266.
Hermann, G., Beck, F. M. and Sheridan, J. F. (1995) Stress-induced glucocorticoid response modulates mononuclear cell trafficking during an experimental influenza viral infection. J. Neuroimmunol., 56, 179-186.
Huang, S., Hendriks, W., Althage, A., Hemmi, S., Bluethmann, H., Kamijo, R., Vilcek, J., Zinkernagel, R. M. and Aguet, M. (1993) Immune response in mice that lack the interferon-gamma receptor. Science, 259, 1742-1745.
Huber, S. A., Kupperman, J. and Newell, M. K. (1999) Hormonal regulation of CD4(+) T-cell responses ini coxsackievirus B3-induced myocarditis in mice. J. Virol., 73, 4689-4695.
Huber, S. A. and Pfaeffle, B. (1994) Differential Th1 and Th2 cell responses in male and female BALB/c mice infected with coxsackievirus group B type 3. J. Virol., 68, 5126-5132.
Imura, H. and Fukata, J. (1994) Endocrine-paracrine interaction in communication between the immune and endocrine systems. Activation of the hypothalamic-pituitary-adrenal axis in inflammation. Eur. J. Endocrinol., 130, 32-37.
Karupiah, G., Blanden, R. V. and Ramshaw, I. A. (1990) Interferon-γ is involved in the recovery of athymic nude mice from recombinant vaccinia virus/interleukin 2 infection. J. Exp. Med., 172, 1495-1503.
Kass, E. H. and Finland, M. (1953) Adrenocortical hormones in infection and immunity. Annu. Rev. Microbiol., 7, 361-388.
Khansari, D. N., Murgo, A. J. and Faith, R. E. (1990) Effects of stress on the immune system. Immunol. Today, 11, 170-175.
Kligman, F. (1951) The effect of cortisone on the pathologic responses of guinea pigs infected cutaneously with fungi, viruses and bacteria. J. Lab. Clin. Med., 37, 615-620.
Labrie, F., Simard, J., Luu-The, V., Pelletier, G., Belanger, A., Lachance, Y., Zhao, H. F., Labrie, C., Breton, N., de Launoit, Y. and et al. (1992) Structure and tissue-specific expression of 3 beta-hydroxysteroid dehydrogenase/5-ene-4-ene isomerase genes in human and rat classical and peripheral steroidogenic tissues. J. Steroid. Biochem. Mol. Biol., 41, 421-435.
Loria, R. M. and Padgett, D. A. (1992) Androstenediol regulates systemic resistance against lethal infections in mice. Arch. Virol., 127, 103-115.
Mackett, M., Smith, G. L. and Moss, B. (1985) The construction and characterization of vaccinia virus recombinants expressing foreign genes. In Glover, D. M. (ed.), DNA Cloning: A Practical Approach. IRL Press, Oxford, Vol. 2, pp. 191-211.
Martel, C., Melner, M. H., Gagne, D., Simard, J. and Labrie, F. (1994) Widespread tissue distribution of steroid sulfatase, 3 beta-hydroxysteroid dehydrogenase/delta 5-delta 4 isomerase (3 beta-HSD), 17 beta-HSD 5 alpha-reductase and aromatase activities in the rhesus monkey. Mol. Cell. Endocrinol., 10⁴, 103-111.
Massung, R. F., Esposito, J. J., Liu, L.-I., Qi, J., Utterback, T. R., knight, J. C., Aubin, L., Yuran, T. E., Parsons, J. M., Loparev, V. N., Selivanov, N. A., Cavallaro, K. F., Kerlavage, A. R., Mahy, B. W. J. and Venter, A. J. (1993) Potential virulence determinants in terminal regions of variola smallpoxvirus genome. Nature, 366, 748-751.
Massung, R. F., Liu, L. I., Qi, J., Knight, J. C., Yuran, T. E., Kerlavage, A. R., Parsons, J. M., Venter, J. C. and Esposito, J. J. (1994) Analysis of the complete genome of smallpox variola major virus strain Bangladesh-1975. Virology, 201, 215-240.
Melkova, Z. and Esteban, M. (1994) Interferon-gamma severely inhibits DNA synthesis of vaccinia virus in a macrophage cell line. Virology, 198, 731-735.
Moore, J. B. and Smith, G. L. (1992) Steroid hormone synthesis by a vaccinia enzyme: a new type of virus virulence factor. EMBO J., 11, 1973-1980, with erratum at EMBO J., 1992, 11, 3490.
Morel, Y., Mebarki, F., Rheaume, E., Sanchez, R., Forest, M. G. and Simard, J. (1997) Structure-function relationships of 3 beta-hydroxysteroid dehydrogenase: contribution made by the molecular genetics of 3 beta-hydroxysteroid dehydrogenase deficiency. Steroids, 62, 176-184.
Moss, B., Carroll, M. W., Wyatt, L. S., Bennink, J. R., Hirsch, V. M., Goldstein, S., Elkins, W. R., Fuerst, T. R., Lifson, J. D., Piatak, M., Restifo, N P., Overwijk, W., Chamberlain, R., Rosenberg, S. A. and Sutter, G. (1996) Host range restricted, non-replicating vaccinia virus vectors as vaccine candidates. Adv. Exp. Med. Biol., 397, 7-13.
Mossman, K., Upton, C., Buller, R. M. and McFadden, G. (1995) Species specificity of ectromelia virus and vaccinia virus interferon-gamma binding proteins. Virology, 208, 762-769.
Muller, U., Steinhoff, U., Reis, L. F., Hemmi, S., Pavlovic, J., Zinkernagel, R. M. and Aguet, M. (1994) Functional role of type I and type II interferons in antiviral defense. Science, 264, 1918-1921.
Padgett, D. A., Loria, R. M. and Sheridan, J. F. (1997) Endocrine regulation of the immune response to influenza virus infection with a metabolite of DHEA-androstenediol. J. Neuroimmunol., 78, 203-211.
Panicali, D., Davis, S. W., Weinberg, R. L. and Paoletti, E. (1983) Construction of live vaccines by using genetically engineered poxviruses: Biological activity of recombinant vaccinia virus expressing influenza virus hemagglutinin. Proc. Natl. Acad. Sci. USA, 80, 5364-5368.
Pelletier, G., Dupont, E., Simard, J., Luu-The, V., Belanger, A. and Labrie, F. (1992) Ontogeny and subcellular localization of 3 beta-hydroxysteroid dehydrogenase (3 beta-HSD) in the human and rat adrenal, ovary and testis. J. Steroid Biochem. Mol. Biol., 43, 451-467.
Perkus, M. E., Goebel, S. J., Davis, S. W., Johnson, G. P., Norton, E. K. and Paoletti, E. (1991) Deletion of 55 open reading frames from the termini of vaccinia virus. Virology, 180, 406-410.
Ramsay, A. J., Ruby, J. and Ramshaw, I. A. (1993) A case for cytokines as effector molecules in the resolution of virus infection. Immunol. Today, 14, 155-157.
Rao, A. R., Prahlad, I., Swaminathan, M. and Lakshmi, A. (1963) Pregnancy and smallpox. J. Indian Med. Assoc., 40, 353-363.
Rao, A. R., Sumkumar, M. S., Kamalakshi, S., Paramasivam, T. V., Parasuraman, T. A. and Shantha, M. (1968) Experimental variola in monkeys. I. Studies on disease enhancing property of cortisone in smallpox. A preliminary study. Indian. J. Med. Res., 56, 1855-1865.
Ruby, J. and Ramshaw, I. (1991) The antiviral activity of immune CD8⁺ T cells is dependent on interferon-gamma. Lymph. Cytokine Res., 10, 353-358.
Ruzek, M. C., Miller, A. H., Opal, S. M., Pearce, B. D. and Biron, C. A. (1997) Characterization of early cytokine responses and an interleukin (IL)-6-dependent pathway of endogenous glucocorticoid induction during murine cytomegalovirus infection. J. Exp. Med., 185, 1185-1192.
Sarmiento, M., Glasebrook, A. L. and Fitch, F. W. (1980) IgG or IgM monoclonal antibodies reactive with different determinants on the molecular complex bearing Lyt 2 antigen block T cell-mediated cytolysis in the absence of complement. J. Immunol., 125, 2665-2672.
Schleimer, R. P., Jacques, A., Shin, H. S., Lichtenstein, L. M. and Plaut, M. (1984) Inhibition of T cell-mediated cytotoxicity by anti-inflammatory steroids. J. Immunol., 132, 266-271.
Schwiebert, L. M., Beck, L. A., Stellato, C., Bickel, C. A., Bochner, B. S., Schleimer, R. P. and Schwiebert, L. A. (1996) Glucocorticosteroid inhibition of cytokine production: relevance to antiallergic actions. J. Allergy Clin. Immunol., 97, 143-152.
Shchelkunov, S. N., Massung, R. F. and Esposito, J. J. (1995) Comparison of the genome DNA sequences of Bangladesh-1975 and India-1967 variola viruses. Virus Res., 36, 107-118.
Shchelkunov, S. N., Totmenin, A. V., Loparev, V. N., Safronov, P. F., Gutorov, V. V., Chizhikov, V. E., Knight, J. C., Parsons, J. M., Massung, R. F. and Esposito, J. J. (2000) Alastrim smallpox variola minor virus genome DNA sequences. Virology, 266, 361-386.
Skinner, M. A., Moore, J. B., Binns, M. M., Smith, G. L. and Boursnell, M. E. (1994) Deletion of fowlpoxvirus homologues of vaccinia virus genes between the 3 beta-hydroxysteroid dehydrogenase (A44L) and DNA ligase (A50R) genes. J. Gen. Virol., 75, 2495-2498.
Smith, G. L., Chan, Y. S. and Howard, S. T. (1991) Nucleotide sequence of 42 kbp of vaccinia virus strain WR from near the right inverted terminal repeat J. Gen. Virol., 72, 1349-1376.
Smith, G. L., Mackett, M and Moss, B. (1983) Infectious vaccinia virus recombinants that express hepatitis B virus surface antigen. Nature, 302, 490-495.
Sroller, V., Kutinova, L., Nemeckova, S., Simonova, V. and Vonka, V. (1998) Effect of 3-beta-hydroxysteroid dehydrogenase gene deletion on virulence and immunogenicity of different vaccinia viruses and their recombinants. Arch. Virol., 143, 1311-1320.
Turner, G. S. (1967) Respiratory infection of mice with vaccinia virus. J. Gen. Virol., 1, 399-402.
Williamson, J. D., Reith, R. W., Jeffrey, L. J., Arrand, J. R. and Mackett, M. (1990) Biological characterization of recombinant vaccinia viruses in mice infected by the respiratory route. J. Gen. Virol., 71, 2761-2767.

All publications and patent applications cited in this specification are incorporated herein by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of vaccinating a subject comprising administering to the subject an immunogenic agent, wherein the immunogenic agent is a recombinant poxvirus, wherein the recombinant poxvirus genome does not comprise a functional gene encoding a 3β-hydroxysteroid dehydrogenase/Δ⁵-Δ⁴isomerase.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. The method of claim 1 wherein the recombinant poxvirus is selected from the group consisting of orthopoxviruses, parapoxviruses, avipoxviruses, suipoxviruses, molluscipoxviruses, and yatapoxviruses.

15. (canceled)

16. (canceled)

17. (canceled)

18. (canceled)

19. A The method of claim 1,
wherein the recombinant poxvirus has no coding sequence encoding a 3β-hydroxysteroid dehydrogenase/Δ⁵-Δ⁴ isomerase; or
wherein the gene encoding the 3β-hydroxysteroid dehydrogenase/Δ⁵-Δ⁴isomerase is disrupted, mutated or truncated such that its gene product has reduced activity; or
wherein one or more mutations or deletions in the promoter or other upstream sequences of the gene encoding a 3β-hydroxysteroid dehydrogenase/Δ⁵-Δ⁴isomerase cause expression of the gene to be compromised, leading to reduced levels of gene expression.

20. (canceled)

21. (canceled)

22. (canceled)

23. A vaccine composition comprising a recombinant poxvirus wherein the poxvirus genome does not comprise a functional 3β-hydroxysteroid dehydrogenase/Δ⁵-Δ⁴isomerase, and a pharmaceutically suitable carrier.

24. The vaccine composition according to claim 23, wherein said composition further comprises one or more additives selected from the group consisting of a preservative, a stabiliser and an adjuvant.

25. The vaccine composition according to claim 23, wherein said recombinant poxvirus is selected from the group consisting of vaccinia viruses, parapoxviruses, avipoxviruses, suipoxviruses, molluscipoxviruses, and yatapoxviruses.

26. (canceled)

27. A vaccine composition according to claim 23,
wherein the recombinant poxvirus has no coding sequence encoding a 3β-hydroxysteroid dehydrogenase/Δ⁵-Δ⁴isomerase; or
wherein the gene encoding the 3β-hydroxysteroid dehydrogenase/Δ⁵-Δ⁴isomerase is disrupted, mutated or truncated such that its gene product has reduced activity or
wherein one or more mutations or deletions in the promoter or other upstream sequences of the gene encoding a 3β-hydroxysteroid dehydrogenase/Δ⁵-Δ⁴isomerase cause expression of the gene to be compromised, leading to reduced levels of gene expression.

28. The method of claim 1, wherein said immunogenic agent is a recombinant poxvirus having a genome comprising a non-poxvirus gene or a fragment of a non-poxvirus gene which gene or fragment encodes an antigen.

29. (canceled)

30. The method of claim 28, wherein the poxvirus is a vaccinia virus, a cowpox virus, a camelpox virus or an ectromelia virus, or a derivative of any of those viruses.

31. (canceled)

32. The method of claim 28, wherein the poxvirus is a vaccinia virus strain selected from the group consisting of Lister, Copenhagen, Wyeth, New York City Board of Health, NYVAC, Praha virus, DRYVAX Wyeth-derived virus, LIVP, IHD-J, IHD-W, Tian Tan, Tashkent, King Institute, Patwadanger, EM-63, Evans, Bern, LC16m0 and MVA.

33. (canceled)

34. The method of claim 28, wherein the non-poxvirus gene or non-poxvirus gene fragment that encodes an antigen is a non-poxvirus gene or non-poxvirus gene fragment against the gene product of which a protective immune response in a subject is desirable.

35. (canceled)

36. (canceled)

37. The method according to claim 28, wherein the administration of said immunogenic agent is for the prophylaxis of an infection caused by a pathogenic agent, or for the prophylaxis or treatment of a disease associated with aberrant cells.

38. The method according to claim 37, in which the non-poxvirus gene encodes an immunogenic peptide or polypeptide of an infectious pathogen, or an antigenic peptide or polypeptide of aberrant cells, the elimination or induced quiescence of which is beneficial.

39. The method according to claim 28, wherein the recombinant poxvirus is selected from the group consisting of parapoxviruses, avipoxviruses, suipoxviruses, molluscipoxviruses and yatapoxviruses.

40. The method according to claim 28, wherein the non-poxvirus gene that encodes an antigen is a non-poxvirus gene against the gene product of which a protective immune response in a subject is desirable.

41. The method according to claim 28,
wherein the poxvirus has no coding sequence encoding a 3β-hydroxysteroid dehydrogenase/Δ⁵-Δ⁴isomerase, or
wherein the gene encoding the 3β-hydroxysteroid dehydrogenase/Δ⁵-Δ⁴isomerase is disrupted, mutated or truncated such that its gene product has reduced activity; or
wherein one or more mutations or deletions in the promoter or other upstream sequences of the gene encoding a 3β-hydroxysteroid dehydrogenase/Δ⁵-Δ⁴isomerase cause expression of the gene to be compromised, leading to reduced levels of gene expression.

42. (canceled)

43. (canceled)

44. A vaccine composition comprising a poxvirus having a genome comprising a non-poxvirus gene or a fragment of a non-poxvirus gene which gene or fragment encodes an antigen, wherein the poxvirus genome does not comprise a functional gene encoding a 3β-hydroxysteroid dehydrogenase/Δ⁵-Δ⁴isomerase, and a pharmaceutically suitable carrier.

45. The vaccine composition according to claim 44, further comprising one or more additives selected from the group consisting of an antibiotic, a preservative, a stabiliser and an adjuvant.

46. The vaccine composition according to claim 44, wherein said poxvirus is selected from the group consisting of a vaccinia virus, a cowpox virus, a camelpox virus, and an ectromelia virus, and derivatives thereof.

47. (canceled)

48. (canceled)

49. (canceled)

50. A recombinant poxvirus having a genome comprising a non-poxvirus gene or a fragment of a non-poxvirus gene which gene or fragment encodes an antigen, wherein the poxvirus genome does not comprise a functional gene encoding a 3β-hydroxysteroid dehydrogenase/Δ⁵-Δ⁴isomerase, with the proviso that the non-poxvirus gene or fragment of a non-poxvirus gene is not a gene encoding varicella-zoster virus glycoprotein E, hepatitis B virus preS2-S protein or E. coli guanine phosphoribosyl transferase

51. The vaccine composition of claim 44, wherein said recombinant poxvirus is a vaccinia virus strain selected from the group consisting of Lister, Copenhagen, Wyeth, New York City Board of Health, NYVAC, Praha virus, DRYVAX Wyeth-derived virus, LIVP, IHD-J, IHD-W, Tian Tan, Tashkent, King Institute, Patwadanger, EM-63, Evans, Bern, LC16m0 or MVA.

52. The vaccine composition of claim 44, wherein said poxvirus is selected from the group consisting of parapoxviruses, avipoxviruses, suipoxviruses, molluscipoxviruses, and yatapoxviruses.

53. The vaccine composition of claim 44,
wherein the recombinant poxvirus has no coding sequence encoding a 3β-hydroxysteroid dehydrogenase/Δ⁵-Δ⁴isomerase; or
wherein the gene encoding the 3β-hydroxysteroid dehydrogenase/Δ⁵-Δ⁴isomerase is disrupted, mutated or truncated such that its gene product has reduced activity; or
wherein one or more mutations or deletions in the promoter or other upstream sequences of the gene encoding a 3β-hydroxysteroid dehydrogenase/Δ⁵-Δ⁴isomerase cause expression of the gene to be compromised, leading to reduced levels of gene expression.
